(19) United States
(12) Patent Application Publication
(10) Pub. No.: US 2026/0020780 A1
(43) Pub. Date: Jan. 22, 2026

(54) RAPID DETECTION OF HYPOGLYCEMIA INCIDENCE USING CONTINUOUS GLUCOSE MONITORING

(71) Applicant: Dexcom, Inc., San Diego, CA (US)

(72) Inventors: Thibault Pierre GAUTIER, Dulles, VA (US); Patricio Hernan COLMEGNA, Charlottesville, VA (US)

(21) Appl. No.: 19/086,766

(22) Filed: Mar. 21, 2025

Related U.S. Application Data
(60) Provisional application No. 63/674,183, filed on Jul. 22, 2024.

(51) Int. Cl. A61B 5/145; A61B 5/00; G16H 10/60

(52) U.S. Cl. CPC A61B 5/14532; A61B 5/7225; A61B 5/7282; A61B 5/742; G16H 10/60

(57) ABSTRACT

Certain aspects of the present disclosure provide systems and techniques for rapid detection of repetitive metabolic events in a host based on measured analyte data provided by an analyte monitor worn by the host. An example system is configured to obtain measured glucose data of the host. A subset of the measured glucose data is determined, based on performing a filtering operation on the measured glucose data. A respective range of a likelihood of an occurrence of a metabolic is determined for each value within the subset of the measured glucose data. For at least one value within the subset of the measured glucose data, a state of the metabolic event is determined based in part on at least one of an upper bound or a lower bound of the respective range corresponding to the at least one value within the subset of the measured glucose data.

### CROSS-REFERENCE TO RELATED APPLICATION

This application claims priority to and benefit of U.S. Provisional Patent Application No. 63/674,183, filed Jul. 22, 2024, which is hereby expressly incorporated by reference herein in its entirety as if fully set forth below and for all applicable purposes.

### BACKGROUND

Diabetes mellitus is a metabolic condition relating to the production or use of insulin by the body. Insulin is a hormone that allows the body to use glucose for energy or to store glucose as fat.

When a person eats a meal that contains carbohydrates, the digestive system absorbs nutrients, ultimately depositing glucose in the person's blood. Blood glucose can be used for energy or stored as fat. The body normally maintains blood glucose levels in a range that provides sufficient energy to support bodily functions and avoids problems that can arise when glucose levels are too high or too low. Regulation of blood glucose levels depends on the production and use of insulin, which regulates the movement of blood glucose into cells.

When the body does not produce enough insulin, or when the body is unable to effectively use insulin that is present, blood sugar levels can elevate beyond normal ranges. The state of having a higher than normal blood sugar level is called “hyperglycemia.” Chronic hyperglycemia can lead to a number of health problems, such as cardiovascular disease, cataract and other eye problems, nerve damage (neuropathy), skin ulcers, and kidney damage. Hyperglycemia can also lead to acute problems, such as diabetic ketoacidosis—a state in which the body becomes excessively acidic due to the production of excess ketones, or body acids. The state of having lower than normal blood glucose levels is called “hypoglycemia.” Severe hypoglycemia can lead to damage of the heart muscle, neurocognitive dysfunction, and in certain cases, acute crises that can result in seizures or even death.

A patient living with diabetes can receive insulin to manage blood glucose levels. Insulin can be received, for example, through a manual injection with a needle. Wearable insulin pumps are also available. Diet and exercise also affect blood glucose levels.

Diabetes conditions are sometimes referred to as “Type 1” and “Type 2”. A Type 1 diabetes patient is typically able to use insulin when it is present, but the body is unable to produce sufficient amounts of insulin, because of a problem with the insulin-producing beta cells of the pancreas. A Type 2 diabetes patient may produce some insulin, but the patient has become “insulin resistant” due to a reduced sensitivity to insulin. The result is that even though insulin is present in the body, the insulin is not sufficiently used by the patient's body to effectively regulate blood sugar levels.

Patients with diabetes can benefit from real-time diabetes management guidance, as determined based on a physiological state of the patient, in order to stay within a target glucose range and avoid physical complications. In certain cases, the physiological state of the patient is determined using monitoring systems that measure glucose levels, which inform the identification and/or prediction of adverse metabolic (e.g., glycemic) events, such as hyperglycemia and hypoglycemia, and the type of guidance provided to the patient.

For example, such monitoring systems may utilize a continuous glucose monitor (CGM) to measure a patient's glucose levels over time. The measured glucose levels may then be processed by the monitoring system to identify and/or predict adverse metabolic events, and/or to provide guidance to the patient for treatment and or actions to abate or prevent the occurrence of such adverse metabolic events. For example, trends, statistics, or other metrics may be derived from the glucose levels and used to identify and/or predict adverse metabolic events. Or, in certain cases, the glucose levels themselves may be used to identify and/or predict adverse metabolic events.

Even with the systems described above, however, the management of diabetes presents many challenges for patients, clinicians, and caregivers, as a confluence of various factors can impact a patient's glucose levels, thus affecting the accuracy of glycemic event prediction and the guidance provided by diagnostics systems.

### SUMMARY

Certain embodiments provide a system. The system includes a memory and a processor communicatively coupled to the memory. The processor is configured to obtain measured glucose data of a host. The processor is also configured to determine a subset of the measured glucose data, based on performing a filtering operation on the measured glucose data. The processor is also configured to determine, for each value within the subset of the measured glucose data, a respective range of a likelihood of an occurrence of a metabolic event. The processor is further configured to determine, for at least one value within the subset of the measured glucose data, a state of the metabolic event based in part on at least one of an upper bound or a lower bound of the respective range corresponding to the at least one value within the subset of the measured glucose data. The processor is further configured to cause an output indicative of the state of the metabolic event to be displayed via a display device associated with the host.

Certain embodiments provide a method. The method includes obtaining measured glucose data of a host. The method also includes determining a subset of the measured glucose data, based on performing a filtering operation on the measured glucose data. The method also includes determining, for each value within the subset of the measured glucose data, a respective range of a likelihood of an occurrence of a metabolic event. The method further includes determining, for at least one value within the subset of the measured glucose data, a state of the metabolic event based in part on at least one of an upper bound or a lower bound of the respective range corresponding to the at least one value within the subset of the measured glucose data. The method further includes causing an output indicative of the state of the metabolic event to be displayed via a display device associated with the host.

Certain embodiments provide a non-transitory computer-readable storage medium. The non-transitory computer-readable storage medium includes computer-executable code which, when executed by one or more processors, perform an operation. The operation includes obtaining measured glucose data of a host. The operation also includes determining a subset of the measured glucose data, based on performing a filtering operation on the measured glucose data. The operation also includes determining, for each value within the subset of the measured glucose data, a respective range of a likelihood of an occurrence of a metabolic event. The operation further includes determining, for at least one value within the subset of the measured glucose data, a state of the metabolic event based in part on at least one of an upper bound or a lower bound of the respective range corresponding to the at least one value within the subset of the measured glucose data. The operation further includes causing an output indicative of the state of the metabolic event to be displayed via a display device associated with the host.

### BRIEF DESCRIPTION OF THE DRAWINGS

So that the manner in which the above-recited features of the present disclosure can be understood in detail, a more particular description, briefly summarized above, may be had by reference to aspects, some of which are illustrated in the drawings. It is to be noted, however, that the appended drawings illustrate only certain typical aspects of this disclosure and are therefore not to be considered limiting of its scope, for the description may admit to other equally effective aspects.

FIG. 1 illustrates aspects of an example health monitoring and support system used in connection with implementing embodiments of the present disclosure.

FIG. 2 is a diagram conceptually illustrating an example continuous analyte monitoring system including example continuous analyte sensor(s) with sensor electronics, according to certain embodiments of the present disclosure.

FIG. 3 illustrates example inputs and example metrics that are calculated based on the inputs for use by the health monitoring and support system of FIG. 1, according to certain embodiments of the present disclosure.

FIG. 4 is a flowchart of an example method for detecting metabolic events in a host, according to certain embodiments of the present disclosure.

FIG. 5A depicts a graph illustrating an estimated range of likelihood of occurrence of a metabolic event for measured glucose data, according to certain embodiments of the present disclosure.

FIG. 5B depicts a graph illustrating states of the metabolic event for the measured glucose data of the graph depicted in FIG. 5A, according to certain embodiments of the present disclosure.

FIG. 6 is a block diagram depicting an example computing device, according to certain embodiments of the present disclosure.

To facilitate understanding, identical reference numerals have been used, where possible, to designate identical elements that are common to the figures. It is contemplated that elements disclosed in one aspect may be beneficially utilized on other aspects without specific recitation.

### DETAILED DESCRIPTION

A major issue with Type 1 diabetes (TID) is the management of hypoglycemia, a condition in which blood glucose levels are low (e.g., glucose levels <70 mg/dl). Low blood glucose levels may cause symptoms in a TID patient such as dizziness, confusion, sweating, weakness, and, in severe cases, loss of consciousness or seizures. TID patients as well as patients with Type 2 diabetes (T2D) may use an analyte monitor (e.g., CGM) to measure their analyte concentration levels over time (e.g., glucose concentration levels during the day, such as every 1 minute, 5 minutes, 10 minutes, etc.). In certain continuous analyte monitoring systems, a transcutaneous continuous analyte sensor that is inserted into the patient is used to monitor the patient's analyte levels, thereby providing analyte measurements reflective of the physiological state of the patient. An analyte may be understood as any substance of interest that is to be measured or is being measured. Examples of such analytes include glucose, ketones, lactate, insulin, electrolytes, creatinine, as well as a number of other biomarkers including proteins, metabolites, and nucleic acids. The continuous analyte sensor may interact with the desired analyte(s), e.g., through aptamers (single-stranded DNA or RNA molecules that bind to a specific analyte). The sensor produces an electric signal (e.g., an electric current or voltage) that a sensor electronics module converts into an analyte concentration. The continuous analyte monitoring system may periodically transmit the analyte measurements to a display device (e.g., CGM display device) for presentation to the patient.

Certain existing analyte monitoring systems may employ machine learning techniques to determine risk of significant metabolic events (e.g., hypoglycemia) occurring in a patient. For example, the machine learning techniques can be utilized to predict, classify, and detect incoming hypoglycemia incident in TID patients. In an illustrative example, after a display device receives a patient's measured analyte data for an extended period of time, such as 10 days, 2 weeks, etc., the display device may analyze the measured analyte data with machine learning techniques to predict an occurrence of a significant metabolic event (e.g., hypoglycemia). Such machine learning techniques can include artificial neural networks (ANNs), support vector machines (SVMs), genetic programming (GP), random forest (RF), hidden Markov models (HMMs), and hybrid and ensemble models, as illustrative examples.

However, one potential drawback to analyte monitoring systems that use machine learning based techniques to predict significant metabolic events is that such techniques may be unable to detect recurrent (e.g., ongoing) significant metabolic events, such as hypoglycemia, promptly. For example, such machine learning based techniques may rely on accumulating a patient's measured analyte data over an extended period of time in order to predict an occurrence of a significant metabolic event, such as hypoglycemia. As a result, existing analyte monitoring systems that use such machine learning based techniques to predict significant metabolic events may be of limited use in helping a host prevent and/or manage such events.

Another potential drawback to analyte monitoring systems that use machine learning based techniques to predict significant metabolic events is that such techniques may involve a significant amount of compute resources (e.g., processor(s), memory, storage, or a combination thereof), which can increase power consumption and reduce battery life of analyte monitors that use such techniques.

Accordingly, the present disclosure describes techniques and systems for detecting metabolic events (e.g., hypoglycemia events) in a host based on measured analyte data provided by an analyte monitor (e.g., CGM) worn by the host in a manner that provides robust protection against analyte monitor artifacts. For example, certain embodiments provide an algorithm that is configured to utilize a host's analyte measurements (e.g., estimated glucose values (EGV) data) to detect when the host is experiencing a significant metabolic event. The algorithm described herein can operate on demand, utilizing the host's current analyte measurements and, if available, the host's prior analyte measurements to detect whether the host has experienced a prior significant metabolic event and/or is experiencing a significant metabolic event. As a result, the algorithm described herein can provide rapid detection of repetitive significant metabolic events, such as repetitive hypoglycemia events, based on a host's previous measured analyte data, the host's current measured analyte data, or a combination thereof.

In certain embodiments, the algorithm determines, for each analyte measurement of a host, a respective range of likelihoods of an occurrence of a metabolic event (e.g., hypoglycemia) for the host. Based on the respective range associated with a given analyte measurement(s), the algorithm can provide an indication of a state of the metabolic event. For example, based on the respective range associated with a given analyte measurement(s), the algorithm can (i) set a “TRUE” flag indicating that occurrence of the metabolic event can be confidently inferred from the analyte measurement(s) (e.g., a first set of predetermined conditions is satisfied), (ii) set a “FALSE” flag indicating that absence of the metabolic event can be confidently inferred from the analyte measurement(s) (e.g., a second set of predetermined conditions is satisfied), or (iii) set an “UNDETERMINED” flag indicating that presence and absence of the metabolic event cannot be confidently inferred from the analyte measurement(s) (e.g., a third set of predetermined conditions is satisfied). Additionally, in certain embodiments, the algorithm indicates the start and end times of the analyzed data segment for any of the three potential states, “TRUE,” “FALSE,” and “UNDETERMINED.”

The techniques and analyte monitoring systems for detecting metabolic events (e.g., hypoglycemia events) in a host described herein may provide various technical advantages. For example, by using the algorithm described herein, analyte monitoring systems may be more successful in detecting repetitive significant metabolic events relative to machine learning based techniques. For instance, the algorithm may more accurately detect significant metabolic events based on measured analyte data from a host, where the measured analyte data is representative of the host's individualized patterns and behaviors (e.g., eating patterns, exercise routines, etc.). The algorithm can be implemented in real-time (e.g., the algorithm can detect in real-time whether a host is experiencing a significant metabolic event) and/or can be used to indicate whether the host has experienced a prior significant metabolic event.

As another example, relative to machine learning based techniques, the algorithm described herein can be implemented with a significantly reduced amount of compute resources without compromising accuracy (e.g., the algorithm effectively mitigates analyte sensor artifacts) and/or detection speed. For instance, the algorithm described herein is capable of operating on a single analyte measurement or small number of analyte measurements (e.g., fewer than 10) in contrast to machine learning based techniques that may require a large number of analyte measurements to be accumulated before an accurate prediction can be made. As a result, the reduced data input size of the algorithm can improve the performance of the algorithm in detecting significant metabolic events, relative to machine learning based techniques. Additionally, the algorithm described herein can improve the speed of detecting metabolic events while lowering processing requirements and ensuring robustness against analyte sensor artifacts, relative to machine learning based techniques.

Although the terms “first,” “second,” “third,” etc., may be used herein to describe various elements, components, regions, layers and/or sections, these elements, components, regions, layers and/or sections should not be limited by these terms. These terms may be only used to distinguish one element, component, region, layer or section from another region, layer, or section. Terms such as “first,” “second,” and other numerical terms, when used herein, do not imply a sequence or order unless clearly indicated by the context. Thus, a first element, component, region, layer, or section discussed herein could be termed a second element, component, region, layer, or section without departing from the teachings of the example embodiments.

As used herein, the term “continuous” analyte monitoring refers to monitoring one or more analytes in a fully continuous, semi-continuous, periodic manner, which results in a data stream of analyte values over time. A data stream of analyte values over time is what allows for meaningful data and insight to be derived using the algorithms described herein for detecting significant metabolic events in a host and providing feedback regarding presence or absence of metabolic events in a host. In other words, single point-in-time measurements collected as a result of a patient visiting their health care professional every few months results in sporadic data points (e.g., that are, at best, months apart in timing) that cannot form the basis of any meaningful data or insight to be derived. As such, without the continuous analyte monitoring system of the embodiments herein, it is simply impossible to continuously monitor for occurrence of significant metabolic events in a host over time, as well as continuously provide feedback related to occurrence of metabolic events, as described herein.

Further, the data stream of analyte values collected over time, with the continuous analyte monitoring system presented herein, include real-time analyte values, which allows for deriving meaningful data and insight in real-time using the systems and algorithms described herein. The derived real-time data and insight in turn allows for providing real-time classification of a host and detection of significant metabolic events, as well as real-time feedback related to occurrence of metabolic events. Real-time analyte values herein refer to analyte values that become available and actionable within seconds or minutes of being produced as a result of at least one sensor electronics module of the continuous analyte monitoring system (1) converting sensor current(s) (i.e., analog electrical signals) generated by the continuous analyte sensor(s) into sensor count values, (2) calibrating the count values to generate at least glucose values (e.g., estimated glucose values (EGV)) and/or other analyte concentration values using calibration techniques described herein to account for the sensitivity of the continuous analyte sensor(s), and (3) transmitting measured glucose values and/or other analyte concentration data to a display device via wireless connection.

For example, the at least one sensor electronics module may be configured to sample the analog electrical signals at a particular sampling period (or rate), such as every 1 second (1 Hz), 5 seconds, 10 seconds, 30 seconds, 1 minute, 3 minutes, 5 minutes, etc., and to transmit the measured glucose values and/or other analyte concentration data to a display device at a particular transmission period (or rate), which may be the same as (or longer than) the sampling period, such as every 1 minute (0.016 Hz), 5 minutes, 10 minutes, etc.

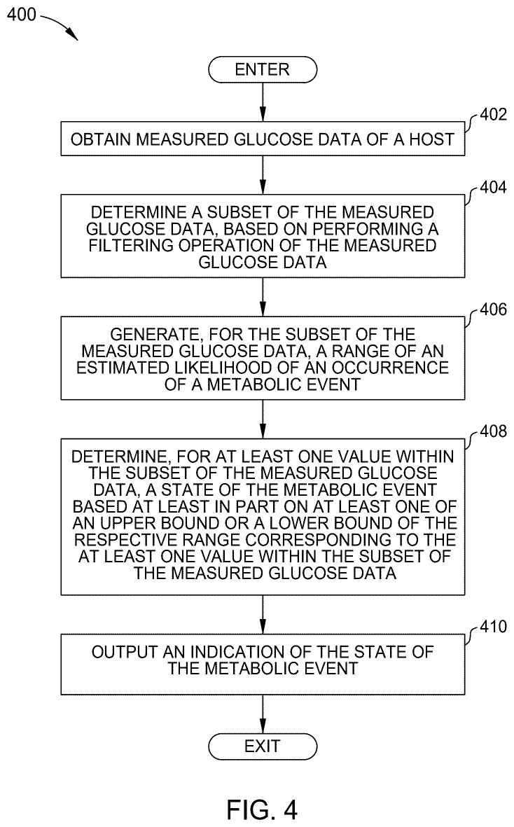

The real-time analyte data that is continuously generated by the continuous analyte monitoring system described herein, therefore, allows the system herein to perform rapid detection of repetitive (e.g., ongoing) significant metabolic events, in real-time and/or retrospectively, which is technically infeasible to perform using existing or conventional techniques or systems. Further, because of the real-time nature of this data, it is also humanly impossible to continuously process a real-time data stream of analyte values over time to derive meaningful data and insight using the algorithms and systems described herein to classify a host and detect significant metabolic events in a host, as well as provide real-time feedback related to occurrence of metabolic events. In other words, deriving meaningful data and insight from a stream of real-time data that is continuously generated, processed, calibrated, and analyzed, using the algorithms and systems described herein, is not a task that can be mentally performed. For example, executing the algorithms described in relation to FIG. 4 in real-time and on a continuous basis, which would involve using a stream of real-time data that is continuously generated by a host's continuous analyte monitoring system and/or using a significantly large amount of population data (e.g., hundreds or thousands of data points for each one of thousands or millions of hosts in the host population), is not a task that can be mentally performed, especially in real-time.

Further, certain embodiments herein are directed to a technical solution to a technical problem associated with analyte sensor systems. In particular, each analyte sensor system that is manufactured by a sensor manufacturer might perform slightly different. As such, there might be inconsistencies between sensors and the measurements the sensors generate once in use. Accordingly, certain embodiments herein are directed to determining the performance of an analyte sensor system during a manufacturing calibration process (in vitro), which includes quantifying certain sensor operating parameters, such as a calibration slope (also known as calibration sensitivity), a calibration baseline, etc.

Generally, calibration sensitivity refers to the amount of electrical current produced by an analyte sensor of an analyte sensor system when immersed in a predetermined amount of a measured analyte. The amount of electrical current may be expressed in units of picoAmps (pA) or counts. The amount of measured analyte may be expressed as a concentration level in units of milligrams per deciliter (mg/dL), and the calibration sensitivity may be expressed in units of pA/(mg/dL) or counts/(mg/dL). The calibration baseline refers to the amount of electrical current produced by the analyte sensor when no analyte is detected, and may be expressed in units of pA or counts.

The calibration sensitivity, calibration baseline, and other information related to the sensitivity profile for the analyte sensor system may be programmed into the sensor electronics module of the analyte sensor system during the manufacturing process, and then used to convert the analyte sensor electrical signals into measured analyte concentration levels. For example, the calibration slope (calibration sensitivity) may be used to predict an initial in vivo sensitivity (M₀) and a final in vivo sensitivity (M_{f}), which are programmed into the sensor electronics module and used to convert the analyte sensor electrical signals into measured analyte concentration levels.

In certain embodiments, during in vivo use, the sensor electronics module of an analyte sensor system samples the analog electrical signals produced by the analyte sensor to generate analyte sensor count values, and then determines the measured analyte concentration levels based on the analyte sensor count values, the initial in vivo sensitivity (M₀), and the final in vivo sensitivity (M_{f}). For example, measured analyte concentration levels may be determined using a sensitivity function M(t) that is based on the initial in vivo sensitivity (M₀) and the final in vivo sensitivity (M_{f}). The sensitivity function M(t) may expressed in several different ways, such as a simple correction factor that is not dependent on elapsed time (tᵢ) of in vivo use, a linear relationship between sensitivity and time (tᵢ), an exponential relationship between sensitivity and time (tᵢ), etc. Equation 1 presents one technique for determining a measured analyte concentration level (ACL) from an analyte sensor count value (count) at a time tᵢ:

$$ACL = count / M ( {t}_{i} ) \qquad Eq . 1$$

A calibration baseline (baseline) may also be used to determine a measured analyte concentration level (ACL) from an analyte sensor count value (count) at a time tᵢ, and Equation 2 presents one technique:

$$ACL = ( count - baseline ) / M ( {t}_{i} ) \qquad Eq . 2$$

### Example System Including an Example Continuous Analyte Sensor for Rapid Detection of Metabolic Events

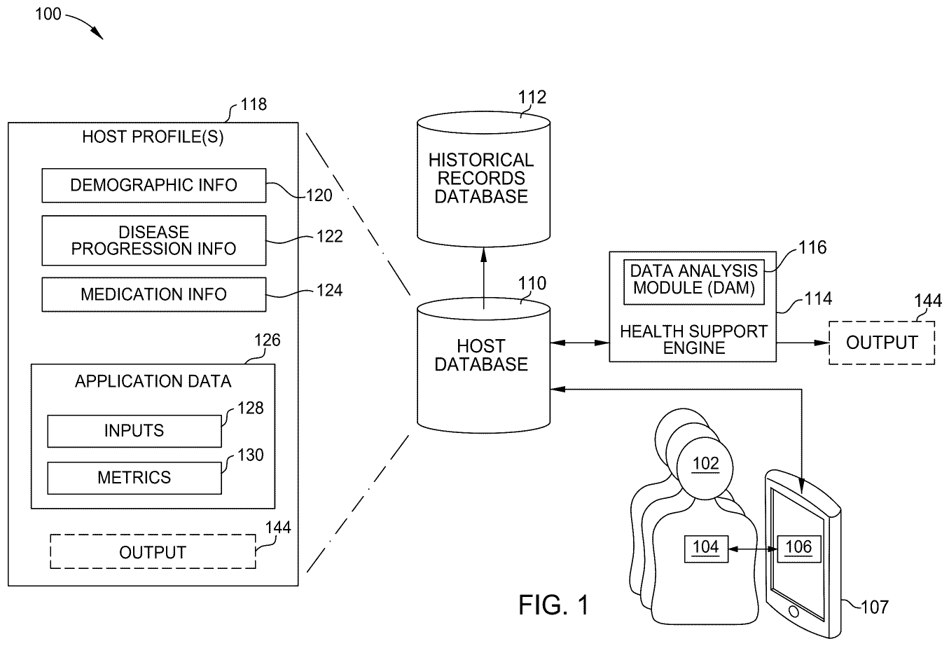

FIG. 1 illustrates an example health monitoring and support system 100 (hereinafter referred to as “system 100”), in accordance with certain embodiments of the disclosure. The system 100 may be utilized for monitoring host health and displaying data related to occurrence of significant metabolic events using various user interfaces to hosts associated with system 100. In certain embodiments, the system 100 may be utilized to detect whether hosts 102 (individually referred to herein as a host and collectively referred to herein as hosts) have experienced a prior significant metabolic event (e.g., hypoglycemia) and/or are experiencing a current significant metabolic event. A host, in certain embodiments, may be a metabolically unfit host, who may suffer from liver issues (e.g., disease, condition, or failure), a host with a metabolic disorder, or any other conditions impacting the metabolic fitness of the host. As discussed herein, metabolic fitness refers to a host's ability to maintain glucose levels in a range that provides sufficient energy to support bodily functions, which, in some cases, could be impacted by the host's pancreas issues (function, disease, failure, etc.) or other metabolic disorders.

In certain embodiments, the system 100 includes continuous analyte monitoring system 104, a display device 107 that executes application 106, a health support engine 114, a host database 110, and a historical records database 112, each of which is described in more detail below.

The term “analyte” as used herein is a broad term used in its ordinary sense, including, without limitation, to refer to a substance or chemical constituent in a biological fluid (for example, blood, interstitial fluid, cerebral spinal fluid, lymph fluid or urine) that can be analyzed. Analytes can include naturally occurring substances, artificial substances, metabolites, and/or reaction products. Analytes for measurement by the devices and methods may include, but may not be limited to, potassium, glucose, endogenous insulin, acarboxyprothrombin; acylcarnitine; endogenous insulin; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); androstenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, glucose-6-phosphate dehydrogenase, hemoglobin A, hemoglobin S, hemoglobin C, hemoglobin D, hemoglobin E, hemoglobin F, D-Punjab, hepatitis B virus, HCMV, HIV-1, HTLV-1, MCAD, RNA, PKU, Plasmodium vivax, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sisomicin; somatomedin C; specific antibodies recognizing any one or more of the following that may include (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin.

Salts, sugar, protein, fat, vitamins, and hormones (e.g., insulin) naturally occurring in blood or interstitial fluids can also constitute analytes in certain implementations. The analyte can be naturally present in the biological fluid, for example, a metabolic product, a hormone, an antigen, an antibody, and the like. Alternatively, the analyte can be introduced into the body or exogenous, for example, a contrast agent for imaging, a radioisotope, a chemical agent, a fluorocarbon-based synthetic blood, or a drug or pharmaceutical composition, including but not limited to insulin; glucagon, ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Pleginc); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA), and intermediaries in the Citric Acid Cycle.

While the analytes that are measured and analyzed by the devices and methods described herein include lactate, glucose, and/or ketones, in some cases other analytes listed above may also be considered.

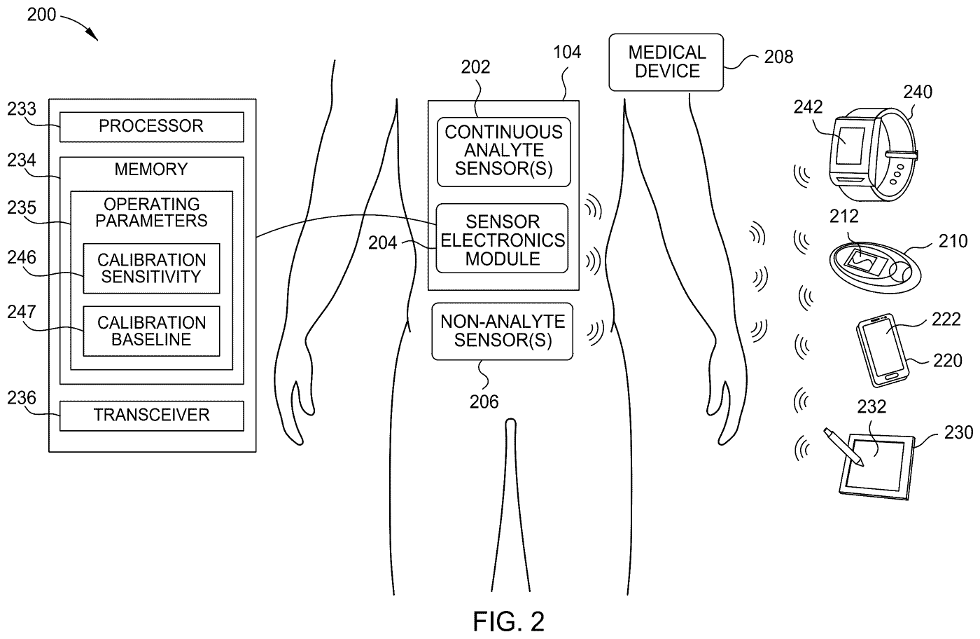

In certain embodiments, continuous analyte monitoring system 104 is configured to continuously measure one or more analytes and transmit the analyte measurements to display device 107 for use by application 106. In certain embodiments, continuous analyte monitoring system 104 transmits the analyte measurements to display device 107 through a wireless connection (e.g., Bluetooth connection). In certain embodiments, display device 107 is a smart phone. However, in certain other embodiments, display device 107 may instead be any other type of computing device such as a laptop computer, a smart watch, a fitness tracker, a cycling computer, a tablet, or any other computing device capable of executing application 106. In certain embodiments, continuous analyte monitoring system 104 may further be configured to directly transmit analyte measurements to another (secondary) display device 107 through a wireless connection (e.g., Bluetooth connection). In such embodiments, the other (secondary) display device 107 may receive analyte measurements provided by continuous analyte monitoring system 104 through the (primary) display device 107. Continuous analyte monitoring system 104 may be described in more detail with respect to FIG. 2.

Application 106 is a mobile health application that is configured to receive and analyze analyte measurements from analyte monitoring system 104. For example, application 106 stores information about a host 102, including the host's analyte measurements, in a host profile 118 of the host 102 for processing and analysis as well as for use by the health support engine 114 to determine and provide data regarding the state of metabolic events, as well as feedback or guidance to the host 102 regarding the occurrence of metabolic events.

Note that, any reference to health support engine 114 providing an indication of the occurrence or absence of a metabolic event, a suggestion, an instruction, or a recommendation to the host 102 can alternatively be automatically provided to the application 106 of display device 107. For example, as described herein, health support engine 114 may provide a notification that the host 102 has experienced a prior metabolic event, a notification that the host 102 is experiencing a current metabolic event, a notification that the host 102 is about to experience a metabolic event, or any combination thereof.

Health support engine 114 refers to a set of software instructions with one or more software modules, including data analysis module (DAM) 116. In certain embodiments, health support engine 114 executes entirely on one or more computing devices in a private or a public cloud. In such embodiments, application 106 communicates with health support engine 114 over a network (e.g., Internet). In certain other embodiments, health support engine 114 executes partially on one or more local devices, such as display device 107, and partially on one or more computing devices in a private or a public cloud. In certain other embodiments, health support engine 114 executes entirely on one or more local devices, such as display device 107. As discussed in more detail herein, health support engine 114 may provide data regarding the state of metabolic events, as well as feedback or guidance to the host 102 regarding the occurrence of metabolic events via application 106. In certain embodiments, health support engine 114 may provide data regarding the state of metabolic events, as well as feedback or guidance to the host 102 regarding the occurrence of metabolic events, based on information included in host profile 118.

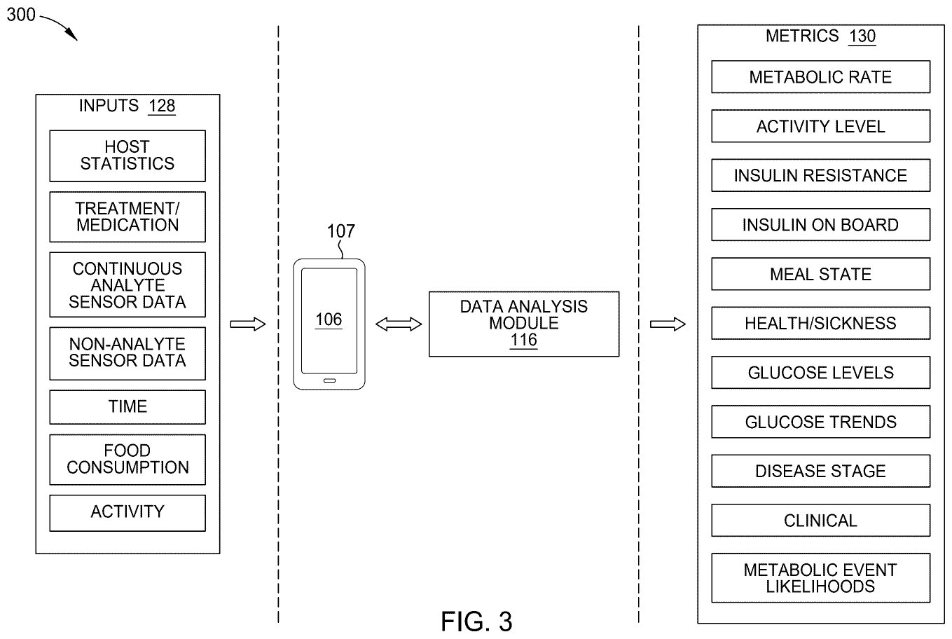

Host profile 118 may include information collected about the host 102 from application 106. For example, application 106 provides a set of inputs 128, including the analyte measurements associated with one or more analytes received from continuous analyte monitoring system 104 that are stored in host profile 118. In certain embodiments, inputs 128 provided by application 106 include other data in addition to analyte measurements. For example, application 106 may obtain additional inputs 128 through manual host input, one or more other non-analyte sensors or devices, other applications executing on display device 107, etc. Non-analyte sensors and devices include one or more of, but are not limited to, an insulin pump, respiratory sensor, sensors or devices provided by display device 107 (e.g., accelerometer, camera, global positioning system (GPS), heart rate monitor, electrocardiogram (ECG), etc.) or other host accessories (e.g., a smart watch, a continuous positive airway pressure (CPAP) machine, or a fitness tracker), or any other sensors or devices that provide relevant information about the host (e.g., sensors on exercise equipment). Inputs 128 of host profile 118 provided by application 106 are described in further detail below with respect to FIG. 3.

DAM 116 of health support engine 114 is configured to process the set of inputs 128 to determine one or more metrics 130. Metrics 130, discussed in more detail below with respect to FIG. 3, may, at least in some cases, be generally indicative of the health or state of a host 102, such as one or more of the physiological state of a host 102, trends associated with the health or state of a host 102, etc. In certain embodiments, metrics 130 may then be used by health support engine 114 as input for providing information and/or feedback regarding the state of a metabolic event to a host 102. As shown, metrics 130 are also stored in host profile 118.

Host profile 118 also includes demographic info 120, disease info 122, and/or medication info 124. In certain embodiments, such information may be provided through host input or obtained from certain data stores (e.g., electronic medical records, etc.). In certain embodiments, demographic info 120 may include one or more of the host's age, BMI (body mass index), ethnicity, gender, etc. In certain embodiments, disease info 122 may include information about one or more diseases of a host 102, including relevant information pertaining to the host's metabolic fitness, liver disease, diabetes, kidney disease, and/or any conditions or diseases relevant to metabolic fitness. In certain embodiments, disease info 122 may also include the length of time since diagnosis, the level of disease control, level of compliance with disease management therapy, other types of diagnoses (e.g., heart disease, obesity), etc. In certain embodiments, disease info 122 may include hospitalizations and/or surgical history. In certain embodiments, disease info 122 may include other measures of health (e.g., heart rate, stress, sleep, etc.) or fitness (e.g., cardiovascular endurance, metabolic state, gait information, muscular strength and/or power, muscular endurance, and other measures of fitness), and/or the like. In certain embodiments, medication info 124 may include information about the amount and type of a medication taken by host 102, such as insulin or non-insulin diabetes medications and/or non-diabetes medication taken by host 102.

In certain embodiments, application 106 may obtain demographic info 120, disease progression info 122, and/or medication info 124 from the host 102 in the form of user input or from other sources. In certain embodiments, host profile 118 is dynamic because at least part of the information that is stored in host profile 118 may be revised or updated over time and/or new information may be added to host profile 118 by health support engine 114 and/or application 106. Accordingly, information in host profile 118 stored in host database 110 provides an up-to-date repository of information related to the host 102.

Host database 110, in certain embodiments, refers to a storage server that operates, for example, in a public or private cloud. Host database 110 may be implemented as any type of datastore, such as relational databases, non-relational databases, key-value datastores, file systems including hierarchical file systems, and the like. In some exemplary implementations, host database 110 is distributed. For example, host database 110 may comprise a plurality of persistent storage devices, which are distributed. Furthermore, host database 110 may be replicated so that the storage devices are geographically dispersed.

Host database 110 includes host profiles 118 associated with a plurality of hosts 102, including hosts 102 who similarly interact or have interacted in the past with application 106 on their own devices. Host profiles 118 stored in host database 110 are accessible to not only application 106, but to health support engine 114 as well. Host profiles 118 in host database 110 may be accessible to application 106 and/or health support engine 114 over one or more networks (not shown), such as one or more wireless networks. As described above, health support engine 114, and more specifically DAM 116 of health support engine 114, can fetch inputs 128 from a host's profile 118 stored in host database 110 and compute one or more metrics 130 which can then be stored as application data 126 in the host's profile 118.

In certain embodiments, host profiles 118 stored in host database 110 may also be stored in historical records database 112. Host profiles 118 stored in historical records database 112 may provide a repository of up-to-date information and historical information for each host 102 of application 106. Thus, historical records database 112 essentially provides all data related to each host 102 of application 106, where data is stored using timestamps. The timestamp associated with any piece of information stored in historical records database 112 may identify, for example, when the piece of information was obtained and/or updated.

Data related to each host 102 stored in historical records database 112 may provide time series data collected over the lifetime of the host. For example, the data may include physiological information (e.g., height and weight), analyte sensor data, as well as non-analyte sensor data (e.g., heart rate, respiratory rate, etc.). Such data may indicate physiological states of the host (e.g., metabolic events, such as hypoglycemia), lactate levels of the host, glucose levels of the host, insulin levels of host, free fatty acid levels of the host, states/conditions of one or more organs of the host, habits of the host (e.g., activity levels, food consumption, etc.), medication prescribed throughout the lifetime of the disease, as well as progress of outcomes such as weight loss and metabolic fitness over time, etc.

Although depicted as separate databases for conceptual clarity, in certain embodiments, host database 110 and historical records database 112 may operate as a single database. That is, historical and current data related to hosts 102 of continuous analyte monitoring system 104 and application 106 may be stored in a single database. The single database may be a storage server that operates in a public or private cloud.

As mentioned previously, system 100 is configured to provide data related to occurrence of significant metabolic events in a host 102 using continuous analyte monitoring system 104, including, at least, a continuous glucose monitor. In certain embodiments, as a part of providing such information, health support engine 114 is configured to provide real-time and or non-real-time notifications regarding the state of metabolic events to the host 102 and/or others, including but not limited to, healthcare providers, family members of the host 102, caregivers of the host 102, etc. The notifications from health support engine 114 may be intended to alert the host 102 and/or others regarding prior, current, and/or future occurrences of significant metabolic events and to allow the host 102 to modify behavior and/or patterns to prevent disease development and/or progression (e.g., loss of consciousness or seizures).

In particular, health support engine 114 may obtain host profile 118 associated with a host 102 and stored in host database 110, use information in host profile 118 as input into an algorithm described herein, and output an indication of a state of a metabolic event for the host 102 (e.g., shown as output 144 in FIG. 1). Output 144 generated by health support engine 114 may also indicate a change in the state of the metabolic event for the host 102 over time. Output 144 may be provided to the host 102 (e.g., through application 106), to a caretaker of the host 102 (e.g., a parent, a relative, a guardian, a teacher, a physical therapist, a fitness trainer, a nurse, etc.), to a physician or healthcare provider of the host 102, or any other individual that has an interest in the wellbeing of the host 102 for purposes of improving the health of the host, such as, in some cases by effectuating recommended treatment. Output 144 generated by health support engine 114 may be stored in host database 110.

FIG. 2 is a diagram 200 conceptually illustrating an example continuous analyte monitoring system 104 including example continuous analyte sensor(s) with sensor electronics, in accordance with certain aspects of the present disclosure. For example, continuous analyte monitoring system 104 may be configured to continuously monitor one or more analytes of a host, in accordance with certain aspects of the present disclosure.

Continuous analyte monitoring system 104 in the illustrated embodiment includes sensor electronics module 204 and one or more continuous analyte sensor(s) 202 (individually referred to herein as continuous analyte sensor 202 and collectively referred to herein as continuous analyte sensors 202) associated with sensor electronics module 204. Sensor electronics module 204 may be in wireless communication (e.g., directly or indirectly) with one or more of display devices 210, 220, 230, and 240. In certain embodiments, sensor electronics module 204 may also be in wireless communication (e.g., directly or indirectly) with one or more medical devices, such as medical devices 208 (individually referred to herein as medical device 208 and collectively referred to herein as medical devices 208), and/or one or more other non-analyte sensors 206 (individually referred to herein as non-analyte sensor 206 and collectively referred to herein as non-analyte sensor 206).

In certain embodiments, a continuous analyte sensor 202 may comprise one or more sensors for detecting and/or measuring analyte(s). The continuous analyte sensor 202 may be a multi-analyte sensor configured to continuously measure two or more analytes or a single analyte sensor configured to continuously measure a single analyte as a non-invasive device, a subcutaneous device, a transcutaneous device, a transdermal device, and/or an intravascular device. In certain embodiments, the continuous analyte sensor 202 may be configured to continuously measure analyte levels of a host using one or more techniques, such as enzymatic techniques, chemical techniques, physical techniques, electrochemical techniques, spectrophotometric techniques, polarimetric techniques, calorimetric techniques, iontophoretic techniques, radiometric techniques, immunochemical techniques, and the like. The term “continuous,” as used herein, can mean fully continuous, semi-continuous, periodic, etc. In certain embodiments, the continuous analyte sensor 202 provides a data stream indicative of the concentration of one or more analytes in the host. The data stream may include raw data signals, which are then converted into a calibrated and/or filtered data stream used to provide estimated analyte value(s) to the host.

In certain embodiments, the continuous analyte sensor 202 may be a multi-analyte sensor, configured to continuously measure multiple analytes in a host's body. For example, in certain embodiments, the continuous multi-analyte sensor 202 may be a single sensor configured to measure lactate, glucose, ketones (e.g., 3-beta-hydroxybutyrate, acetoacetate, acetone, etc.), glycerol, and/or free fatty acids in the host's body.

In certain embodiments, one or more multi-analyte sensors may be used in combination with one or more single analyte sensors. As an illustrative example, a multi-analyte sensor may be configured to continuously measure lactate and glucose and may, in some cases, be used in combination with an analyte sensor configured to measure only ketones or only potassium. Information from each of the multi-analyte sensor(s) and single analyte sensor(s) may be combined to provide detection of significant metabolic events using methods described herein. In further embodiments, other non-contact and or periodic or semi-continuous, but temporally limited, measurements for physiological information may be integrated into the system such as by including weight scale information or non-contact heart rate monitoring from a sensor pad under the host while in a chair or bed, through an infra-red camera detecting temperature and/or blood flow patterns of the host, and/or through a visual camera with machine vision for height, weight, or other parameter estimation without physical contact.

In certain embodiments, the continuous analyte sensor(s) 202 may comprise a percutaneous wire that has a proximal portion coupled to the sensor electronics module 204 and a distal portion with several electrodes, such as a measurement electrode and a reference electrode. The measurement (or working) electrode may be coated, covered, treated, embedded, etc., with one or more chemical molecules that react with a particular analyte, and the reference electrode may provide a reference electrical voltage. The measurement electrode may generate the analog electrical signal, which is conveyed along a conductor that extends from the measurement electrode to the proximal portion of the percutaneous wire that is coupled to the sensor electronics module 204. After the continuous analyte monitoring system 104 has been applied to epidermis of the patient, continuous analyte sensor(s) 202 penetrates the epidermis, and the distal portion extends into the dermis and/or subcutaneous tissue under epidermis. Other configurations of continuous analyte sensor(s) 202 may also be used, such as a multi-analyte sensor that includes multiple measurement electrodes, each generating an analog electrical signal that represents the concentration levels of a particular analyte.

Generally, a single-analyte sensor generates an analog electrical signal that is proportional to the concentration level of a particular analyte. Similarly, each multi-analyte sensor generates multiple analog electrical signals, and each analog electrical signal is proportional to the concentration level of a particular analyte. As an illustrative example, continuous analyte sensor 202 may include a single-analyte sensor configured to measure lactate concentration levels, and another single-analyte sensor configured to measure glucose concentration levels of the patient. As another illustrative example, continuous analyte sensor(s) 202 may include a single-analyte sensor configured to measure lactate concentration levels, and one or more multi-analyte sensors configured to measure glucose concentration levels, ketone concentration levels, creatinine concentration levels, etc. As yet another illustrative example, continuous analyte sensor(s) 202 may include a multi-analyte sensor configured to measure lactate concentration levels, glucose concentration levels, ketone concentration levels, creatinine concentration levels, etc. Accordingly, continuous analyte sensor(s) 202 is configured to generate at least one analog electrical signal that is proportional to the concentration level of a particular analyte, and sensor electronics module 204 is configured to convert the analog electrical signal into an analyte sensor count values, calibrate the analyte sensor count values based on the sensitivity profile of the continuous analyte sensor(s) 202 to generate measured analyte concentration levels, and transmit the measured analyte concentration level data, including the measured analyte concentration levels, to a display device, such as display devices 210, 220, and/or 230, via a wireless connection. For example, sensor electronics module 204 may be configured to sample the analog electrical signal at a particular sampling period (or rate), such as every 1 second (1 Hz), 5 seconds, 10 seconds, 30 seconds, 1 minute, 3 minutes, 5 minutes, etc., and to transmit the measured analyte concentration data to the display device at a particular transmission period (or rate), which may be the same as (or longer than) the sampling period, such as every 1 minute (0.016 Hz), 5 minutes, 10 minutes, 30 minutes, at the conclusion of the wear period, etc. Depending on the sampling and transmission periods, the measured analyte concentration data transmitted to the display device include at least one measured analyte concentration level having an associated time tag, sequence number, etc.

In certain embodiments, continuous analyte sensor(s) 202 may incorporate a thermocouple within, or alongside, the percutaneous wire to provide an analog temperature signal to the sensor electronics module 204, which may be used to correct the analog electrical signal or the measured analyte data for temperature. In other embodiments, the thermocouple may be incorporated into the sensor electronics module 204 above the adhesive pad, or, alternatively, the thermocouple may contact the epidermis of the patient through openings in the adhesive pad.

In certain embodiments, the sensor electronics module 204 includes, inter alia, processor 233, storage element or memory 234, wireless transmitter/receiver (transceiver) 236, one or more antennas coupled to wireless transceiver 236, analog electrical signal processing circuitry, analog to-digital (A/D) signal processing circuitry, digital signal processing circuitry, a power source for continuous analyte sensor(s) 202 (such as a potentiostat), etc.

Processor 233 may be a general-purpose or application-specific microprocessor, an application-specific integrated circuit (ASIC), a field programmable gate array (FPGA), etc., that executes instructions to perform control, computation, input/output, etc, functions for the sensor electronics module 204. Processor 233 may include a single integrated circuit, such as a micro processing device, or multiple integrated circuit devices and/or circuit boards working in cooperation to accomplish the appropriate functionality. In certain embodiments, processor 233, memory 234, wireless transceiver 236, the A/D signal processing circuitry, and the digital signal processing circuitry may be combined into a system-on-chip (SoC).

Generally, processor 233 may be configured to sample the analog electrical signal using the A/D signal processing circuitry at regular intervals (such as the sampling period) to generate analyte sensor count values based on the analog electrical signals produced by the continuous analyte sensor(s) 202, calibrate the analyte sensor count values based on the sensitivity profile of the continuous analyte sensor(s) 202 to generate measured analyte concentration levels, and generate measured analyte data from the measured analyte concentration levels, generate sensor data packages that include, inter alia, the measured analyte concentration level data. Processor 233 may store the measured analyte concentration level data in memory 234, and generate the sensor data packages at regular intervals (such as the transmission period) for transmission by wireless transceiver 236 to a display device, such as display devices 210, 220, 230, and/or 240. Processor 233 may also add additional data to the sensor data packages, such as supplemental sensor information that includes a sensor identifier, a sensor status, temperatures that correspond to the measured analyte data, etc. The sensor data packages are then wirelessly transmitted over a wireless connection to the display device. In certain embodiments, the wireless connection is a Bluetooth or Bluetooth Low Energy (BLE) connection. In such embodiments, the sensor data packages are transmitted in the form of Bluetooth or BLE data packets to the display device

In various embodiments, memory 234 may include volatile and nonvolatile medium. For example, memory 234 may include combinations of random access memory (RAM), dynamic RAM (DRAM), static RAM (SRAM), read only memory (ROM), flash memory, cache memory, and/or any other type of non-transitory computer-readable medium. Memory 234 may store one or more analyte sensor system applications, modules, instruction sets, etc. for execution by processor 233, such as instructions to generate measured analyte data from the analyte sensor count values, etc.

Memory 234 may also store certain sensor operating parameters 235, such as a calibration slope (or calibration sensitivity), a calibration baseline, etc. In particular, the calibration sensitivity, calibration baseline, and other information related to the sensitivity profile for the sensor electronics module 204 may be programmed into the sensor electronics module 204 during the manufacturing process, and then used to convert the analyte sensor electrical signals into measured analyte concentration levels. For example, as discussed above, the calibration slope may be used to predict an initial in vivo sensitivity (M₀) and a final in vivo sensitivity (M_{f}), which are stored in memory 234 and used to convert the analyte sensor electrical signals into measured analyte concentration levels. In certain embodiments, calibration sensitivity (Mcc) 246 and/or calibration baseline 247 may be stored in memory 234.

In certain embodiments, sensor electronics module 204 includes electronic circuitry associated with measuring and processing the continuous analyte sensor data, including prospective algorithms associated with processing and calibration of the sensor data. Sensor electronics module 204 can be physically connected to continuous analyte sensor(s) 202 and can be integral with (non-releasably attached to) or releasably attachable to continuous analyte sensor(s) 202. Sensor electronics module 204 may include hardware, firmware, and/or software that enable measurement of levels of analyte(s) via continuous analyte sensor(s) 202. For example, sensor electronics module 204 can include a potentiostat, a power source for providing power to the sensor, other components useful for signal processing and data storage, and a telemetry module for transmitting data from the sensor electronics module to, e.g., one or more display devices. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, and/or a processor.

Display devices 210, 220, 230, and/or 240 are configured for displaying displayable sensor data, including analyte data, which may be transmitted by sensor electronics module 204. Each of display devices 210, 220, 230, or 240 may include a display such as a touchscreen display 212, 222, 232, and/or 242 for displaying sensor data to a host and/or for receiving inputs from the host. For example, a graphical user interface (GUI) may be presented to the host 102 for such purposes. In certain embodiments, the display devices may include other types of user interfaces such as a voice user interface instead of, or in addition to, a touchscreen display for communicating sensor data to the host of the display device and/or for receiving host inputs. Display devices 210, 220, 230, and 240 may be examples of display device 107 illustrated in FIG. 1 used to display sensor data to a host 102 of the system of FIG. 1 and/or to receive input from the host 102.

In certain embodiments, one, some, or all of the display devices are configured to display or otherwise communicate (e.g., verbalize) the sensor data as it is communicated from the sensor electronics module (e.g., in a customized data package that is transmitted to display devices based on their respective preferences), without any additional prospective processing required for calibration and real-time display of the sensor data.

The plurality of display devices may include a custom display device specially designed for displaying certain types of displayable sensor data associated with analyte data received from sensor electronics module. In certain embodiments, the plurality of display devices may be configured for providing alerts/alarms based on the displayable sensor data. Display device 210 is an example of such a custom device. In certain embodiments, one of the plurality of display devices is a smartphone, such as display device 220 which represents a mobile phone, using a commercially available operating system (OS), and configured to display a graphical representation of the continuous sensor data (e.g., including current and historic data). Other display devices can include other hand-held devices, such as display device 230 which represents a tablet, display device 240 which represents a smart watch or fitness tracker, medical device 208 (e.g., an insulin delivery device or a blood glucose meter), and/or a desktop or laptop computer (not shown).

Because different display devices provide different user interfaces, content of the data packages (e.g., amount, format, and/or type of data to be displayed, alarms, and the like) can be customized (e.g., programmed differently by the manufacture and/or by an end host) for each particular display device. Accordingly, in certain embodiments, a plurality of different display devices can be in direct wireless communication with a sensor electronics module (e.g., such as an on-skin sensor electronics module 204 that is physically connected to continuous analyte sensor(s) 202) during a sensor session to enable a plurality of different types and/or levels of display and/or functionality associated with the displayable sensor data.

As mentioned, sensor electronics module 204 may be in communication with a medical device 208. Medical device 208 may be a passive device in some example embodiments of the disclosure. For example, medical device 208 may be an insulin pump for administering insulin to a host 102. For a variety of reasons, it may be desirable for such an insulin pump to receive and track lactate, glucose, ketones, glycerol and free fatty acid values transmitted from continuous analyte monitoring systems 104, where continuous analyte sensor 202 is configured to measure lactate, glucose, ketones, glycerol, and/or free fatty acids.

Further, as mentioned, sensor electronics module 204 may also be in communication with other non-analyte sensors 206. Non-analyte sensors 206 may include, but are not limited to, an altimeter sensor, an accelerometer sensor, a global positioning system (GPS) sensor, a temperature sensor, a respiration rate sensor, etc. Non-analyte sensors 206 may also include monitors such as heart rate monitors, blood pressure monitors, pulse oximeters, caloric intake monitors, indirect calorimetry devices and medicament delivery devices. One or more of these non-analyte sensors 206 may provide data to health support engine 114 described further below. In certain embodiments, a host may manually provide some of the data for processing by health support engine 114 of FIG. 1.

In certain embodiments, non-analyte sensors 206 may further include sensors for measuring skin temperature, core temperature, sweat rate, and/or sweat composition.

In certain embodiments, the non-analyte sensors 206 may be combined in any other configuration, such as, for example, combined with one or more continuous analyte sensors 202. As an illustrative example, a non-analyte sensor, e.g., a temperature sensor, may be combined with a continuous lactate sensor 202 to form a lactate/temperature sensor used to transmit sensor data to the sensor electronics module 204 using common communication circuitry. As another illustrative example, a non-analyte sensor, e.g., a temperature sensor, may be combined with a multi-analyte sensor 202 configured to measure lactate and glucose to form a lactate/glucose/temperature sensor used to transmit sensor data to the sensor electronics module 204 using common communication circuitry.

In certain embodiments, a wireless access point (WAP) may be used to couple one or more of continuous analyte monitoring system 104, the plurality of display devices, medical device(s) 208, and/or non-analyte sensor(s) 206 to one another. For example, a WAP may provide Wi-Fi and/or cellular connectivity among these devices. Near Field Communication (NFC) and or Bluetooth may also be used among devices depicted in diagram 200 of FIG. 2.

FIG. 3 illustrates example inputs and example metrics that are calculated based on the inputs for use by the system 100 of FIG. 1, according to some embodiments disclosed herein. In particular, FIG. 3 provides a more detailed illustration of example inputs and example metrics introduced in FIG. 1.

FIG. 3 illustrates example inputs 128 on the left, application 106 and DAM 116 in the middle, and metrics 130 on the right. In certain embodiments, each one of metrics 130 may correspond to one or more values, e.g., discrete numerical values, ranges, or qualitative values (high/medium/low, stable/unstable, etc.). Some or all of metrics 130 may include time-series data and/or be provided in the form of time-series data. Application 106 obtains inputs 128, which may be in the form of time-series data, through one or more channels (e.g., manual host input, sensors, other applications executing on display device 107, an electronic medical record (EMR) system, etc.). As mentioned previously, in certain embodiments, inputs 128 may be processed by DAM 116 to output a plurality of metrics, such as metrics 130. Inputs 128, metrics 130, or any combination thereof, may be used by health support engine 114 for detecting occurrence of significant metabolic events in a host 102 and other functionalities described herein.

In certain embodiments, starting with inputs 128, host statistics, such as one or more of age, height, weight, BMI, body composition (e.g., % body fat or % muscle from a computed tomography (CT) scan, a magnetic resonance imaging (MRI) scan, dual-energy X-ray absorptiometry (DEXA) scan, etc.), stature, build, or other information may also be provided as an input. In certain embodiments, host statistics are provided through a user interface, by interfacing with an electronic source such as an EMR, and/or from measurement devices. In certain embodiments, the measurement devices include one or more wireless devices, e.g., Bluetooth-enabled, weight scale and/or camera, which may, for example, communicate with the display device 107 to provide host data.

In certain embodiments, treatment/medication information is also provided as an input. Medication information may include information about the type, dosage, and/or timing of when one or more medications are to be taken by the host. For example, the user's medication intake may include the user's insulin delivery. Such information may be received, via a wireless connection on a smart pen, via user input, and/or from an insulin pump (e.g., medical device 108). Insulin delivery information may include one or more of insulin volume, time of delivery, etc. Other configurations, such as insulin action time or duration of insulin action, may also be received as inputs. Treatment information may include information regarding different lifestyle habits recommended by the host's physician. For example, the host's physician may recommend a host follow specific diet recommendations, exercise for a minimum of thirty minutes a day, or adjust insulin dose to in order to put glucose levels in a desired range. In certain embodiments, treatment/medication information may be provided through manual host input.

In certain embodiments, analyte sensor data may also be provided as input, for example, through continuous analyte monitoring system 104 and/or in any of the ways described with respect to FIGS. 1-2. An example of analyte data is glucose data, which may be provide and/or stored as a time series corresponding to time-stamped glucose measurements over time. Other types of analyte data, such as ketone data, potassium data, lactate data, etc., may similarly be provided and/or stored as a time series.

In certain embodiments, input may also be received from one or more non-analyte sensors, such as non-analyte sensors 206 described with respect to FIG. 2. Input from such non-analyte sensors 206 may include information related to a heart rate, a respiration rate, oxygen saturation, blood pressure, or a body temperature (e.g. to detect illness, physical activity, etc.) of a host. In certain embodiments, electromagnetic sensors may also detect low-power radio frequency (RF) fields emitted from objects or tools touching or near the object, which may provide information about host activity or location.

In certain embodiments, input received from non-analyte sensors may include input relating to a host's insulin delivery. In particular, input related to the host's insulin delivery may be received, via a wireless connection on a smart pen, via host input, and/or from an insulin pump. Insulin delivery information may include one or more of insulin volume, time of delivery, etc. Other parameters, such as exogenous insulin action time or duration of exogenous insulin action, may also be received as inputs.

In certain embodiments, starting with inputs 128, food consumption information may include information about one or more of meals, snacks, and/or beverages, such as one or more of the size, content (carbohydrate, fat, protein, etc.), sequence of consumption, and time of consumption. In certain embodiments, food consumption may be provided by a host through manual entry, by providing a photograph through an application that is configured to recognize food types and quantities, and/or by scanning a bar code or menu. In various examples, meal size may be manually entered as one or more of calories, quantity (“three cookies”), menu items (“Royale with Cheese”), and/or food exchanges (1 fruit, 1 dairy). In some examples, meal information may be received via a convenient user interface provided by application 106.

In certain embodiments, food consumption information (the type of food (e.g., liquid or solid, snack or meal, etc.) and/or the composition of the food (e.g., carbohydrate, fat, protein, etc.)) may be determined automatically based on information provided by one or more sensors. Some example sensors may include body sound sensors (e.g., abdominal sounds may be used to detect the types of meal, e.g., liquid/solid food, snack/meal, etc.), radio-frequency sensors, cameras, hyperspectral cameras, and/or analyte (e.g., insulin, glucose, lactate, etc.) sensors to determine the type and/or composition of the food.

In certain embodiments, activity information is also provided as an input. Activity information may be provided, for example, the one or more non-analyte sensors 206 of FIG. 2. In certain embodiments, activity information may additionally be provided through manual input by host 102. Activity information may include, for example, a time series for each of heart rate, activity minutes, step count, floors climbed, location information (e.g., GPS data), calories burned, sleep duration and/or quality, activity level (e.g., light, medium, or heavy), and/or similar information. In addition, or alternatively, the activity information can include one or more time series for recorded activities of one or more defined activity types (e.g., walk, run, sprint, swim, weightlift etc.), where each activity is associated with a duration and/or time period.

In certain embodiments, time may also be provided as an input, such as time of day or time from a real-time clock. For example, in certain embodiments, input analyte data may be timestamped to indicate a date and time when the analyte measurement was taken for the host.

Host input of any of the above-mentioned inputs 128 may be provided through continuous analyte sensor system 104, non-analyte sensors 206, and/or a user interface, such a user interface of display device 107 of FIG. 1. As described above, in certain embodiments, DAM 116 determines or computes the host's metrics 130 based on inputs 128. An example list of metrics 130 is shown in FIG. 3.

In certain embodiments, metabolic rate is a metric that may indicate or include a basal metabolic rate (e.g., energy consumed at rest) and/or an active metabolism (e.g., energy consumed by activity, such as physical exertion). In some examples, basal metabolic rate and active metabolism may be tracked as separate outcome metrics. In certain embodiments, the metabolic rate may be calculated by DAM 116 based on one or more of inputs 128, such as one or more of activity information, analyte sensor data, non-analyte sensor data, time, etc. In certain embodiments, the metabolic rate may be calculated and metabolic rates calculated over time may be time-stamped and stored in the host's profile 118.

In certain embodiments, the activity level metric may indicate the host's level of activity. For example, the activity level may indicate whether the user is exercising, at rest, sleeping, etc. The activity level metric be determined, for example based on input from an activity sensor or other physiologic sensors, such as non-analyte sensors 206. In certain embodiments, the activity level metric may be calculated by DAM 116 based on one or more of inputs 128, such as one or more of activity information, non-analyte sensor data (e.g., accelerometer data), time, host input, etc. In certain embodiments, the activity level may be expressed as a step rate of the host. Activity level metrics may be time-stamped so that they can be correlated with the host's glucose levels at the same time.

In certain embodiments, the metrics 130 include an insulin resistance metric (also referred to herein as “insulin resistance”). The insulin resistance metric may be determined using historical data, real-time data, or a combination thereof, and may, for example, be based upon one or more inputs 128, such as one or more of food consumption information, blood glucose information, insulin delivery information, the resulting glucose levels, etc.

In certain embodiments, the metrics 130 include an insulin on board metric. The insulin on board metric may be determined using insulin delivery information, and/or known or learned (e.g., from patient data) insulin time action profiles, which may account for both basal metabolic rate (e.g., update of insulin to maintain operation of the body) and insulin usage driven by activity or food consumption.

In certain embodiments, the metrics 130 include a meal state metric. The meal state metric may indicate the state the host is in with respect to food consumption. For example, the meal state may indicate whether the host is in one of a fasting state, pre-meal state, eating state, post-meal response state, or stable state. In certain embodiments, the meal state may also indicate nourishment on board, e.g., meals, snacks, or beverages consumed, and may be determined, for example from food consumption information, time of meal information, and/or digestive rate information, which may be correlated to food type, quantity, and/or sequence (e.g., which food/beverage was eaten first.).

In certain embodiments, the metrics 130 include health and sickness metrics. Health and sickness metrics may be determined, for example, based on one or more of host input (e.g., pregnancy information or known sickness information), from non-analyte sensor(s) 206, such as physiologic sensors (e.g., temperature), activity sensors, or a combination thereof. In certain embodiments, based on the values of the health and sickness metrics, for example, the host's state may be defined as being one or more of healthy, ill, rested, or exhausted. In certain embodiments, health and sickness metric may indicate the host's heart rate, stress level, etc.

In certain embodiments, the metrics 130 include analyte level metrics (e.g., glucose level metrics). Analyte level metrics may be determined from analyte data (e.g., glucose measurements obtained from analyte sensor system 104). In some examples, an analyte level metric may also be determined, for example, based upon historical information about analyte levels in particular situations, e.g., given a combination of food consumption, insulin, and/or activity. An analyte level metric may include a rate of change of the analyte, time in range, time spent below a threshold level, time spent above a threshold level, or the like. In certain embodiments, an analyte trend (e.g., glucose trend) may be determined based on the analyte level over a certain period of time. As described above, example analytes may include glucose, ketones, lactate, potassium and others described herein.

In certain embodiments, the metrics 130 include a disease stage. For example, disease stages for Type II diabetics may include a pre-diabetic stage, an oral treatment stage, and a basal insulin treatment stage. In certain embodiments, degree of glycemic control (not shown) may also be determined as an outcome metric, and may be based, for example, on one or more of glucose levels, variation in glucose level, or insulin dosing patterns.

In certain embodiments, the metrics 130 include clinical metrics. Clinical metrics generally indicate a clinical state a host is in with respect to one or more conditions of the host, such as diabetes. For example, in the case of diabetes, clinical metrics may be determined based on glycemic measurements, including one or more of A1C, trends in A1C, time in range, time spent below a threshold level, time spent above a threshold level, and/or other metrics derived from glucose values. In certain embodiments, clinical metrics may also include one or more of estimated A1C, glycemic variability, hypoglycemia, and/or health indicator (time magnitude out of target zone). For example, in certain embodiments, the clinical metrics may include a certain amount of glucose values (e.g., p^{th} EGV percentile) that are below a predetermined threshold (e.g., glucose threshold, gₜₕ) associated with occurrence of a metabolic event (e.g., hypoglycemia).

In certain embodiments, the metrics 130 include metabolic event likelihoods. As described in greater detail herein, the metabolic event likelihoods generally refer to likelihoods (or percentages) of occurrence of a metabolic event, such as hypoglycemia. In certain embodiments, the metabolic event likelihoods may include one or more values, e.g., discrete numerical values, ranges, or qualitative values (high/medium/low, stable/unstable, etc.). As described in greater detail herein, the metabolic event likelihoods may be determined at least in part on one or more of the clinical metrics.

FIG. 4 illustrates an example flowchart of a method 400 for detecting metabolic events (e.g., hypoglycemia events) in a host (e.g., host 102) based on measured analyte data provided by an analyte monitoring system (e.g., continuous analyte monitoring system 104) worn by the host, in accordance with certain embodiments of the disclosure. In certain embodiments, method 400 can be executed, for example, by the health support engine 114. In addition or alternatively, in certain embodiments, the method 400 can be executed, for example, by the application 106. In addition or alternatively, in certain embodiments, the method 400 can be executed generally by any of the display devices 210, 220, 230 and/or 240 of FIG. 2. In addition or alternatively, in certain embodiments, the method 400 can be executed by one or more computing devices in a cloud computing environment. Although any number of systems, in whole or in part, can implement the method 400, to simply discussion, the method 400 will be described primarily in relation to the health support engine 114.

Method 400 may enter at block 402, where the health support engine 114 obtains measured glucose data (e.g., analyte data) of a host 102. The measured glucose data may include previous measured glucose data of the host 102, current measured glucose data of the host 102, or a combination thereof. For example, in certain embodiments, the health support engine 114 may obtain a history of the host's measured glucose data from a storage system, such as host database 110, historical records database 112, or any combination thereof, as illustrative examples. Additionally or alternatively, in certain other embodiments, the health support engine 114 may receive current measured glucose data of the host 102 from a continuous analyte monitoring system 104 (e.g., CGM sensor) worn by the host 102. Note, in certain examples, the current measured glucose data may be obtained directly from the continuous analyte monitoring system 104. In certain other examples, the current measured glucose data may be obtained via a storage system (e.g., host database 110) (e.g., the continuous analyte monitoring system 104 may store the current measured glucose data in the storage system, and the data may be retrieved by the health support engine 114 and/or another system).

In certain embodiments, as new measured glucose data is obtained, the health support engine 114 may create and/or update a history of the measured glucose data with the new measured glucose data, e.g., in the host profile 118. In some cases, the health support engine 114 may sort and de-duplicate the history to maintain a desired (or target) nₕᵢₛₜ days (e.g., 3 days, 5 days, 14 days or more) of data. Note, the value of nₕᵢₛₜ may be based in part on a target performance for the detection of the significant metabolic event, such as hypoglycemia. For example, higher values of nₕᵢₛₜ may be associated with higher confidences for the detection of the significant metabolic event, and lower values of nₕᵢₛₜ may be associated with lower confidences for the detection of the significant metabolic event.

At block 404, the health support engine 114 determines a subset of the measured glucose data (e.g., filtered glucose data, such as filtered EGV data), based on performing a filtering operation on the measured glucose data. In certain embodiments, the filtering operation includes a centered median filter. Note, however, that this is merely an example and any suitable filter consistent with the functionality described herein may be used to determine the subset of the measured glucose data.

In embodiments where a centered median filter is utilized at block 404, the health support engine 114 may apply the centered median filter with n samples to the measured glucose data to obtain the subset of measured glucose data. Assuming the measured glucose data includes a set of EGV data {EGVᵢ} with i={0, . . . , h} and the centered median filter is implemented with a positive odd integer n, the filtered EGV data {f EGVᵢ} is computed using Equation 3 as follows:

$$Eq . 3$$

$${fEGV}_{i} = \{ \begin{matrix}median ( \{ {EGV}_{0} , {EGV}_{1} , … , {EGV}_{i + ⌊ \frac{n}{2} ⌋} \} ) & if i < ⌊ \frac{n}{2} ⌋ \\ median ( \{ {EGV}_{i - ⌊ \frac{n}{2} ⌋} , … , {EGV}_{i} , … , {EGV}_{h} \} ) & if i < ⌊ \frac{n}{2} ⌋ > h \\ median ( \{ {EGV}_{i - ⌊ \frac{n}{2} ⌋} , … , {EGV}_{i} , … , {EGV}_{i + ⌊ \frac{n}{2} ⌋} \} ) & otherwise\end{matrix}$$

$$with ⌊ \frac{n}{2} ⌋ being the floor of \frac{n}{2} .$$

The filtering operation performed in block 404 may smooth the measured glucose data, removing erroneous glucose values due to inaccurate readings or signal loss due to temporary CGM sensor malfunction, poor connection quality, and pressure-induced sensor attenuation, among other causes. For example, the centered median filter is generally a sliding window that computes a median value (fEGVᵢ) for each EGV within the set of EGV data. In Equation 3, n is a smoothing parameter that corresponds to the width of the window that is being smoothed. Larger values of n may correspond to higher amounts of smoothing, whereas smaller values of n may correspond to lower amounts of smoothing. In some cases, the value of n may be based in part on a sampling frequency of the measured glucose data. For example, higher values of n may be used for higher sampling frequencies, since higher sampling frequencies may lead to noisier signals. Likewise, lower values of n may be used for lower sampling frequencies.

At block 406, the health support engine 114 generates, for each value within the subset of the measured glucose data, a respective range of a likelihood of an occurrence of a metabolic event (e.g., metabolic event likelihood metrics of metrics 130 depicted in FIG. 3).

In certain embodiments, generating the ranges in block 406 may involve computing the p^{th} EGV percentile (represented as pEGV) for the subset of measured glucose data, where the p^{th} EGV percentile is the p^{th} percentile amount of EGV values below a predetermined threshold (e.g., glucose threshold, gₜₕ) associated with occurrence of the metabolic event (e.g., hypoglycemia). In certain embodiments, the health support engine 114 computes a default 2^{nd} EGV percentile (e.g., p=2) for the subset of measured glucose data. Note, however, that the health support engine 114 can be configured to use any value of p in order to regulate sensitivity or specificity as needed.

In certain embodiments, generating the ranges in block 406 may further involve, after computing the p^{th} EGV percentile for the subset of measured glucose data, estimating the uncertainty (pₑᵣᵣₒᵣ) around the computed percentile as a function of the amount of data available using the following error model in Equation 4:

$${p}_{error} ( {n}_{exp} , {n}_{egv} ) = \frac{{n}_{exp}}{{n}_{EGV}} ( \frac{{g}_{error}}{{n}_{EGV}} + {b}_{error} ) \qquad Eq . 4$$

where nₑₓₚ and n_{EGV} are the number of expected and gathered EGV points, respectively, and gₑᵣᵣₒᵣ and bₑᵣᵣₒᵣ are the gain and offset of the error model, respectively. Note, the techniques herein may utilize any suitable error model, including parametric error models and nonparametric error models as illustrative examples. The number of expected EGV points (nₑₓₚ) is an estimate of the amount of measured glucose data that is expected within the analyzed data segment. In cases where the number of gathered EGV points (n_{EGV}) is less than the number of expected EGV points (nₑₓₚ) (e.g., due to a variety of factors, such as an unreliable CGM sensor), the value of the uncertainty may increase.

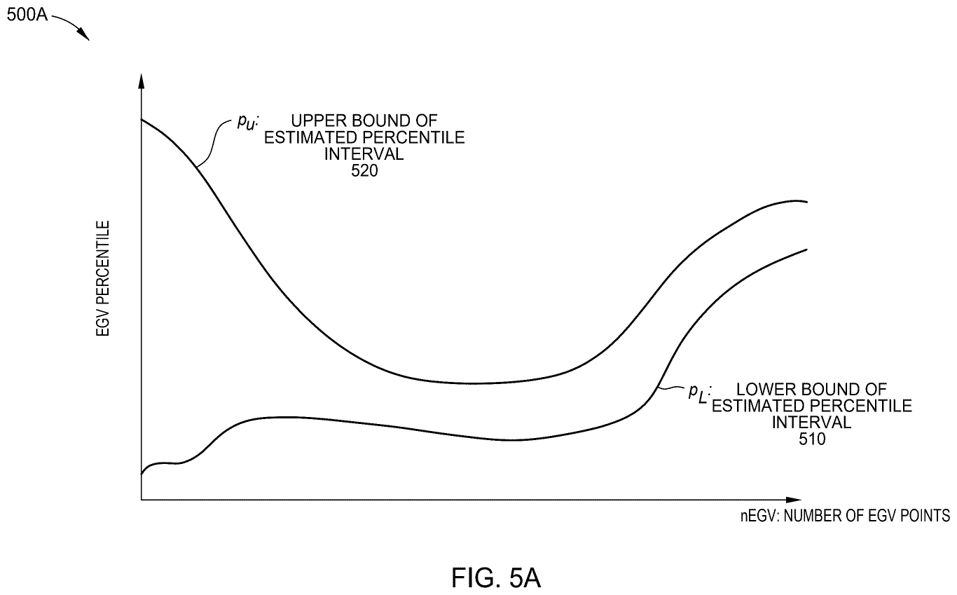

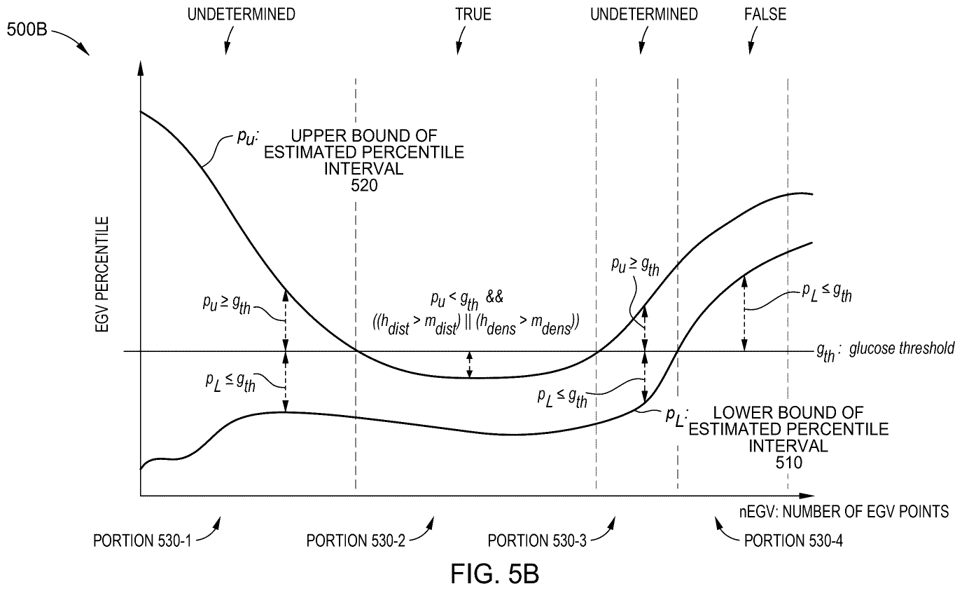

In certain embodiments, generating the ranges in block 406 may further involve, after computing the uncertainty (pₑᵣᵣₒᵣ) around the computed p^{th} EGV percentile for the subset of measured glucose data, computing the range of the uncertainty around the computed p^{th} EGV percentile for the subset of measured glucose data. For example, the range of the uncertainty around the computed p′ EGV percentile for the subset of measured glucose data may have a lower bound (pₗ) and an upper bound (pᵤ) which may be computed using the following Equations 5 and 6, respectively:

$${p}_{l} ( pEGP , {p}_{error} ) = pEGV - {f}_{l} {p}_{error} ( {n}_{exp} , {n}_{egv} ) \qquad Eq . 5$$

$${p}_{u} ( pEGP , {p}_{error} ) = pEGV + {f}_{u} {p}_{error} ( {n}_{exp} , {n}_{egv} ) \qquad Eq . 6$$

where fₗ and fᵤ are asymmetric factors. By way of example, FIG. 5A depicts a graph 500A illustrating an example lower bound (pₗ) 510 and an upper bound (pᵤ) 520 of an estimated range of uncertainty around the computed p^{th} EGV percentile for a subset of measured glucose data, in accordance with certain embodiments of the present disclosure.

In certain embodiments, the values of the asymmetric factors fₗ and fᵤ in Equations 5 and 6, respectively, may be set to achieve a desired sensitivity for the detection of the metabolic event. For example, lower values of fᵤ and fₗ may increase the likelihood of determining a “TRUE” or “FALSE” state for the metabolic event and decrease the likelihood of determining an “UNDETERMINED” state for the metabolic event. On the other hand, higher values of fᵤ and fₗ may decrease the likelihood of determining a “TRUE” or “FALSE” state for the metabolic event and increase the likelihood of determining an “UNDETERMINED” state for the metabolic event.

Referring back to FIG. 4, at block 408, the health support engine 114 determines, for at least one value within the subset of the measured glucose data, a state of the metabolic event based in part on at least one of an upper bound (e.g., pᵤ) or a lower bound (e.g., pₗ) of the respective range corresponding to the at least one value within the subset of the measured glucose data.

In certain embodiments, the health support engine 114, at block 408, may determine the state of the metabolic event is one of “TRUE,” “FALSE,” and “UNDETERMINED” based on one or more respective conditions. That is, the health support engine 114 may determine whether a respective set of conditions associated with a “TRUE” state of the metabolic event, a “FALSE” state of the metabolic event, or an “UNDETERMINED” state of the metabolic event is satisfied, and set the state of the metabolic event to one of “TRUE,” “FALSE,” or “UNDETERMINED” depending on which respective set of conditions is satisfied.

The “first” set of conditions associated with the “TRUE” state of the metabolic event includes: (i) pᵤ<gₜₕ and (ii) h_{dist}>m_{dist}, where h_{dist} is the distance between the first EGV sample (or point) below the glucose threshold (gₜₕ) and the last EGV sample (or point) below the glucose threshold (gₜₕ), and m_{dist} is a predetermined minimum distance between first and last EGV samples below the glucose threshold (8th).

In certain embodiments, the m_{dist} parameter is used to mitigate false detections of metabolic events due to malfunctions of the continuous analyte monitoring system 104 (including, for example, malfunctions of the continuous analyte sensor 202, malfunctions of the sensor electronics module 204, or a combination thereof), such as those that may occur near the end of life of the continuous analyte sensor 202. For example, by imposing the condition that the metabolic events are distant (e.g., h_{dist} is greater than m_{dist}) before declaring a “TRUE” state for the metabolic event, the health support engine 114 can determine with greater confidence that the two metabolic events do not belong to the same time period in which the continuous analyte monitoring system 104 was malfunctioning.

In certain embodiments, in addition to or as an alternative to the m_{dist} parameter, the health support engine 114 may compute and use a density parameter (m_{dens}) to mitigate false detections of metabolic events due to malfunctions of the continuous analyte monitoring system 104. For example, in such embodiments, the “first” set of conditions associated with the “TRUE” state of the metabolic event may include: (i) pᵤ<gₜₕ and at least one of (ii) h_{dist}>m_{dist} Or (iii) h_{dens}>m_{dens}. By way of example, in certain embodiments, h_{dens} may be determined using the following Equation 7:

$${h}_{dens} = \frac{1}{h \times {h}_{max}} {\sum }_{i = 0}^{h} min ( {h}_{max} , {\sum }_{k = i - w}^{i + w} {EGV}_{k} < {g}_{th} ) \qquad Eq . 7$$

where h is the number of available EGV samples, w is a predefined integer (e.g., w=288 or some other value), hₘₐₓ is a predefined integer (e.g., hₘₐₓ=4 or some other value), and m_{dens} is the upper bound of the h_{dens} computed from EGV data from analyte monitors worn by users that are not expected to be experiencing a metabolic event (e.g., hypoglycemia).

Compared to using the m_{dlist} parameter alone, the m_{dens} parameter may provide a higher level of confidence that the two metabolic events are not associated with a malfunctioning continuous analyte monitoring system 104 (including malfunction of the continuous analyte sensor 202, malfunction of the sensor electronics module 204, or a combination thereof). For example, in general, a continuous analyte sensor 202 may be less reliable on “Day 1” operation and at end of life. Thus, in scenarios where the time between the “Day 1” operation and the end of life is greater than m_{dist}, the m_{dist} parameter may not be sufficient to prevent false detection of the metabolic event. However, by computing and evaluating a density parameter (m_{dens}, which is the minimum number of events that have to occur within a time period) before declaring a “TRUE” state for the metabolic event, the health support engine 114 can prevent the scenario of the false detection of the metabolic event due to unreliable operation of the continuous analyte sensor 202 on “Day 1” and at the sensor's end of life.

In certain embodiments, the “second” set of conditions associated with the “FALSE” state of the metabolic event includes pₗ<pᵤ<gₜₕ. In certain embodiments, the “third” set of conditions associated with the “UNDETERMINED” state of the metabolic event includes pᵤ≥gₜₕ and pₗ≤gₜₕ.

By way of example, FIG. 5B depicts a graph 500B illustrating states of the metabolic event for different portions of the EGV points in graph 500A of FIG. 5A, in accordance with certain embodiments of the present disclosure. In particular, graph 500B in FIG. 5B illustrates the conditions that trigger each potential output: TRUE, FALSE, or UNDETERMINED.

As shown in FIG. 5B, the health support engine 114 may output an “UNDETERMINED” flag for a portion 530-1 of EGV points in which the range of the uncertainty around the computed p^{th} EGV percentile for the portion 530-1 of EGV points overlaps the glucose threshold (gₜₕ). In certain illustrative examples, the portion 530-1 of EGV points may be representative of a beginning portion in time in which measured glucose values associated with a host 102 are initially obtained from a continuous analyte monitoring system 104 worn by the host 102. For example, the beginning portion in time may be associated with “Day 1” operation of the continuous analyte monitoring system 104.

As also shown, the health support engine 114 outputs a “TRUE” flag for a subsequent portion 530-2 of EGV points in which (i) the range of the uncertainty around the computed p^{th} EGV percentile for the portion 530-2 of EGV points is below the glucose threshold (gₜₕ) and (ii) at least one of h_{dist}>m_{dist} or h_{dens}>m_{dens}. In certain illustrative examples, in response to outputting the “TRUE” flag, the health support engine 114 may generate and transmit an alert to a display device (e.g., any one of display devices 210, 220, 230, and/or 240 illustrated in FIG. 2) associated with the host 102 and/or to a computing system associated with a healthcare provider (HCP) for the host 102. The alert, for example, may be transmitted to notify the host 102 about the occurrence of the metabolic event and/or prompt the host 102 to change treatment and/or host behavior.

As also shown, the health support engine 114 outputs an “UNDETERMINED” flag for a subsequent portion 530-3 of EGV points in which the range of the uncertainty around the computed p^{th} EGV percentile for the third portion of EGV points overlaps the glucose threshold (8th). In certain illustrative examples, the portion 530-3 of EGV points may be associated with a transition in host behavior and/or treatment. For example, in response to the previous alert, the host 102 may be more careful about avoiding the metabolic event and may have initiated a change in the host's behavior (e.g., increase physical activity) and/or reduced their insulin amount, among other changes.

Lastly, as shown in FIG. 5B, the health support engine 114 outputs a “FALSE” flag for a subsequent portion 530-4 in which the range of the uncertainty around the computed p^{th} EGV percentile for the portion 530-4 of EGV points is above the glucose threshold (gₜₕ). In certain illustrative examples, the portion 530-4 of EGV points may be associated with a completed change in the host's behavior and/or treatment.

Note, although not shown, the health support engine 114 may detect a change in the state of the metabolic event from “FALSE” to “UNDETERMINED.” In such scenarios, the health support engine 114 may generate and transmit an alert notifying the host 102 that they may be approaching occurrence of the metabolic event.

Referring back to FIG. 4, at block 410, the health support engine 114 outputs an indication of the state of the metabolic event. For example, the health support engine 114 may provide an indication of the state of the metabolic event to a display device (e.g., any one of display devices 210, 220, 230, and/or 240 illustrated in FIG. 2) for display to the host 102. In certain embodiments, the health support engine 114 may cause an output indicative of the state of the metabolic event to be displayed to the host 102 via the display device.

Advantageously, the algorithm described herein can provide a more accurate detection of ongoing significant metabolic events (e.g., hypoglycemia) based on measured glucose data from a host, where the measured glucose data is representative of the host's individualized patterns and behaviors. Additionally, the algorithm described herein can be integrated into a cloud-based platform, requiring minimal computational resources, relative to machine learning based techniques. For example, the lightweight nature of the algorithm does not compromise accuracy; rather, it provides a robust alternative for detecting recurring significant metabolic events, such as hypoglycemia. Moreover, as noted, the algorithm described herein remains insensitive to well-known real-world artifacts (e.g., inaccurate glucose readings due to CGM sensor end of life) without compromising detection speed.

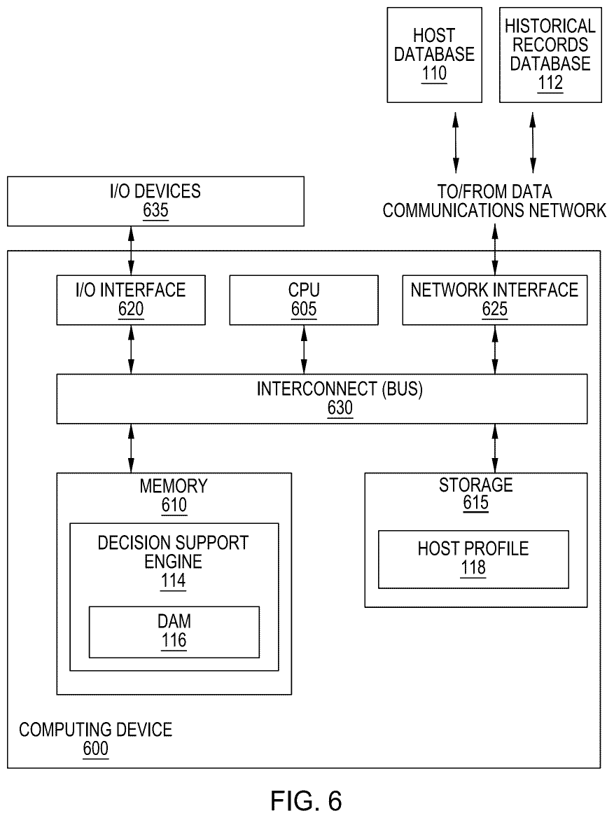

FIG. 6 is a block diagram depicting a computing device 600 configured to execute a health support engine (e.g., health support engine 114), according to certain embodiments disclosed herein. Although depicted as a single physical device, in embodiments, computing device 600 may be implemented using virtual device(s), and/or across a number of devices, such as in a cloud environment. As illustrated, computing device 600 includes a processor 605, memory 610, storage 615, a network interface 625, and one or more I/O interfaces 620. In the illustrated embodiment, processor 605 retrieves and executes programming instructions stored in memory 610, as well as stores and retrieves application data residing in storage 615. Processor 605 is generally representative of a single CPU and/or GPU, multiple CPUs and/or GPUs, a single CPU and/or GPU having multiple processing cores, and the like. Memory 610 is generally included to be representative of a random-access memory. Storage 615 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, input and output (I/O) devices 635 (such as keyboards, monitors, etc.) can be connected via the I/O interface(s) 620. Further, via network interface 625, computing device 600 can be communicatively coupled with one or more other devices and components, such as host database 110 and historical records database 112, as illustrative examples. In certain embodiments, computing device 600 is communicatively coupled with other devices via a network, which may include the Internet, local network(s), and the like. The network may include wired connections, wireless connections, or a combination of wired and wireless connections. As illustrated, processor 605, memory 610, storage 615, network interface(s) 625, and I/O interface(s) 620 are communicatively coupled by one or more interconnects 630. In certain embodiments, computing device 600 is representative of display device 107 associated with the host. In certain embodiments, as discussed above, the display device 107 can include the host's laptop, computer, smartphone, and the like. In another embodiment, computing device 600 is a server executing in a cloud environment.

In the illustrated embodiment, storage 615 includes host profile 118. Memory 610 includes health support engine 114, which itself includes DAM 116.

### Example Embodiments

Implementation examples are described in the following numbered clauses:

Clause 1: A system comprising: a memory; and a processor communicatively coupled to the memory, the processor configured to: obtain measured glucose data of a host; determine a subset of the measured glucose data, based on performing a filtering operation on the measured glucose data; determine, for each value within the subset of the measured glucose data, a respective range of a likelihood of an occurrence of a metabolic event; determine, for at least one value within the subset of the measured glucose data, a state of the metabolic event based in part on at least one of an upper bound or a lower bound of the respective range corresponding to the at least one value within the subset of the measured glucose data; and cause an output indicative of the state of the metabolic event to be displayed via a display device associated with the host.

Clause 2: The system of Clause 1, wherein to determine the state of the metabolic event, the processor is configured to: determine whether a respective set of conditions associated with each of a first state, a second state, or a third state is satisfied; and set the state of the metabolic event to one of the first state, the second state, and the third state based on which respective set of conditions is satisfied.

Clause 3: The system of Clause 2, wherein: the first state is associated with presence of the metabolic event; and the set of conditions associated with the first state comprises the upper bound of the respective range being less than a first threshold.

Clause 4: The system of any one of Clauses 2-3, wherein: the processor is further configured to determine at least one of a distance parameter or a density parameter, based on the subset of the measured glucose data; and the set of conditions associated with the first state further comprises at least one of (i) the distance parameter being greater than a second threshold or (ii) the density parameter being greater than a third threshold.

Clause 5: The system of Clause 4, wherein the distance parameter is a distance between (i) a first value within the subset of the measured glucose data that is below the first threshold and (ii) a second value within the subset of the measured glucose data that is below the first threshold.

Clause 6: The system of any one of Clauses 4-5, wherein the density parameter comprises a number of values within the subset of the measured glucose data that is below the first threshold within a predetermined time period.

Clause 7: The system of any one of Clauses 2-6, wherein: the second state is associated with an absence of the metabolic event; and the set of conditions associated with the second state comprises the lower bound being greater than a threshold.

Clause 8: The system of any one of Clauses 2-7, wherein: the third state is associated with a presence of the metabolic event and an absence of the metabolic event being undetermined; and the set of conditions associated with the third state comprises (i) the upper bound being greater than or equal to a threshold and (ii) the lower bound being less than or equal to the threshold.

Clause 9: The system of any one of Clauses 1-8, wherein to obtain the measured glucose data, the processor is configured to obtain the measured glucose data from a storage system, the measured glucose data comprising historical glucose measurements.

Clause 10: The system of any one of Clauses 1-9, wherein to obtain the measured glucose data, the processor is configured to receive the measured glucose data from a continuous analyte sensor worn by the host, the measured glucose data comprising real-time glucose measurements.

Clause 11: The system of any one of Clauses 1-10, wherein performing the filtering operation comprises applying a centered median filter on the measured glucose data.

Clause 12: A method comprising: obtaining measured glucose data of a host; determining a subset of the measured glucose data, based on performing a filtering operation on the measured glucose data; determining, for each value within the subset of the measured glucose data, a respective range of a likelihood of an occurrence of a metabolic event; determining, for at least one value within the subset of the measured glucose data, a state of the metabolic event based in part on at least one of an upper bound or a lower bound of the respective range corresponding to the at least one value within the subset of the measured glucose data; and causing an output indicative of the state of the metabolic event to be displayed via a display device associated with the host.

Clause 13: The method of Clause 12, wherein determining the state of the metabolic event comprises: determining whether a respective set of conditions associated with each of a first state, a second state, or a third state is satisfied; and setting the state of the metabolic event to one of the first state, the second state, and the third state based on which respective set of conditions is satisfied.

Clause 14: The method of Clause 13, wherein: the first state is associated with presence of the metabolic event; and the set of conditions associated with the first state comprises the upper bound of the respective range being less than a first threshold.

Clause 15: The method of any one of Clauses 13-14, further comprising determining at least one of a distance parameter or a density parameter, based on the subset of the measured glucose data, wherein the set of conditions associated with the first state further comprises at least one of (i) the distance parameter being greater than a second threshold or (ii) the density parameter being greater than a third threshold.

Clause 16: The method of Clause 15, wherein the distance parameter is a distance between (i) a first value within the subset of the measured glucose data that is below the first threshold and (ii) a second value within the subset of the measured glucose data that is below the first threshold.

Clause 17: The method of any one of Clauses 15-16, wherein the density parameter comprises a number of values within the subset of the measured glucose data that is below the first threshold within a predetermined time period.

Clause 18: The method of any one of Clauses 13-17, wherein: the second state is associated with an absence of the metabolic event; and the set of conditions associated with the second state comprises the lower bound being greater than a threshold.

Clause 19: The method of any one of Clauses 13-18, wherein: the third state is associated with a presence of the metabolic event and an absence of the metabolic event being undetermined; and the set of conditions associated with the third state comprises (i) the upper bound being greater than or equal to a threshold and (ii) the lower bound being less than or equal to the threshold.

Clause 20: The method of any one of Clauses 12-19, wherein obtaining the measured glucose data comprises obtaining the measured glucose data from a storage system, the measured glucose data comprising historical glucose measurements.

Clause 21: The method of any one of Clauses 12-20, wherein obtaining the measured glucose data comprises receiving the measured glucose data from a continuous analyte sensor worn by the host, the measured glucose data comprising real-time glucose measurements.

Clause 22: The method of any one of Clauses 12-21, wherein performing the filtering operation comprises applying a centered median filter on the measured glucose data.

Clause 23: A non-transitory computer-readable storage medium comprising computer-executable code, which when executed by one or more processors, perform an operation comprising: obtaining measured glucose data of a host; determining a subset of the measured glucose data, based on performing a filtering operation on the measured glucose data; determining, for each value within the subset of the measured glucose data, a respective range of a likelihood of an occurrence of a metabolic event; determining, for at least one value within the subset of the measured glucose data, a state of the metabolic event based in part on at least one of an upper bound or a lower bound of the respective range corresponding to the at least one value within the subset of the measured glucose data; and causing an output indicative of the state of the metabolic event to be displayed via a display device associated with the host.

Clause 24: The non-transitory computer-readable storage medium of Clause 23, wherein determining the state of the metabolic event comprises: determining whether a respective set of conditions associated with each of a first state, a second state, or a third state is satisfied; and setting the state of the metabolic event to one of the first state, the second state, and the third state based on which respective set of conditions is satisfied.

Clause 25: The non-transitory computer-readable storage medium of Clause 24, wherein: the first state is associated with presence of the metabolic event; and the set of conditions associated with the first state comprises the upper bound of the respective range being less than a first threshold.

Clause 26: The non-transitory computer-readable storage medium of any one of Clauses 24-25, the operation further comprising determining at least one of a distance parameter or a density parameter, based on the subset of the measured glucose data, wherein the set of conditions associated with the first state further comprises at least one of (i) the distance parameter being greater than a second threshold or (ii) the density parameter being greater than a third threshold.

Clause 27: The non-transitory computer-readable storage medium of Clause 26, wherein the distance parameter is a distance between (i) a first value within the subset of the measured glucose data that is below the first threshold and (ii) a second value within the subset of the measured glucose data that is below the first threshold.

Clause 28: The non-transitory computer-readable storage medium of any one of Clauses 26-27, wherein the density parameter comprises a number of values within the subset of the measured glucose data that is below the first threshold within a predetermined time period.

Clause 29: The non-transitory computer-readable storage medium of any one of Clauses 24-28, wherein: the second state is associated with an absence of the metabolic event; and the set of conditions associated with the second state comprises the lower bound being greater than a threshold.

Clause 30: The non-transitory computer-readable storage medium of any one of Clauses 24-29, wherein: the third state is associated with a presence of the metabolic event and an absence of the metabolic event being undetermined; and the set of conditions associated with the third state comprises (i) the upper bound being greater than or equal to a threshold and (ii) the lower bound being less than or equal to the threshold.

Clause 31: The non-transitory computer-readable storage medium of any one of Clauses 23-30, wherein obtaining the measured glucose data comprises obtaining the measured glucose data from a storage system, the measured glucose data comprising historical glucose measurements.

Clause 32: The non-transitory computer-readable storage medium of any one of Clauses 23-31, wherein obtaining the measured glucose data comprises receiving the measured glucose data from a continuous analyte sensor worn by the host, the measured glucose data comprising real-time glucose measurements.

Clause 33: The non-transitory computer-readable storage medium of any one of Clauses 23-32, wherein performing the filtering operation comprises applying a centered median filter on the measured glucose data.

### ADDITIONAL CONSIDERATIONS

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, “a processor,” “at least one processor,” or “one or more processors” generally refer to a single processor configured to perform one or multiple operations or multiple processors configured to collectively perform one or more operations. In the case of multiple processors, performance of the one or more operations could be divided amongst different processors, though one processor may perform multiple operations, and multiple processors could collectively perform a single operation. Similarly, “a memory,” “at least one memory,” or “one or more memories” generally refer to a single memory configured to store data and/or instructions or multiple memories configured to collectively store data and/or instructions.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.”

While various examples of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various example examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described example examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular example of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The term “comprising as used herein is synonymous with “including.” “containing,” or “characterized by” and is inclusive or open-ended and does not exclude additional, unrecited elements or method steps.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific examples and examples described herein, but rather to also cover all modification and alternatives coming with the true scope and spirit of the invention.

1. A system comprising:
a memory; and
a processor communicatively coupled to the memory, the processor configured to:
obtain measured glucose data of a host;
determine a subset of the measured glucose data, based on performing a filtering operation on the measured glucose data;
determine, for each value within the subset of the measured glucose data, a respective range of a likelihood of an occurrence of a metabolic event;
determine, for at least one value within the subset of the measured glucose data, a state of the metabolic event based in part on at least one of an upper bound or a lower bound of the respective range corresponding to the at least one value within the subset of the measured glucose data; and
cause an output indicative of the state of the metabolic event to be displayed via a display device associated with the host.

2. The system of claim 1, wherein to determine the state of the metabolic event, the processor is configured to:
determine whether a respective set of conditions associated with each of a first state, a second state, or a third state is satisfied; and
set the state of the metabolic event to one of the first state, the second state, and the third state based on which respective set of conditions is satisfied.

3. The system of claim 2, wherein:
the first state is associated with presence of the metabolic event; and
the set of conditions associated with the first state comprises the upper bound of the respective range being less than a first threshold.

4. The system of claim 3, wherein:
the processor is further configured to determine at least one of a distance parameter or a density parameter, based on the subset of the measured glucose data; and
the set of conditions associated with the first state further comprises at least one of (i) the distance parameter being greater than a second threshold or (ii) the density parameter being greater than a third threshold.

5. The system of claim 4, wherein the distance parameter is a distance between (i) a first value within the subset of the measured glucose data that is below the first threshold and (ii) a second value within the subset of the measured glucose data that is below the first threshold.

6. The system of claim 4, wherein the density parameter comprises a number of values within the subset of the measured glucose data that is below the first threshold within a predetermined time period.

7. The system of claim 2, wherein:
the second state is associated with an absence of the metabolic event; and
the set of conditions associated with the second state comprises the lower bound being greater than a threshold.

8. The system of claim 2, wherein:
the third state is associated with a presence of the metabolic event and an absence of the metabolic event being undetermined; and
the set of conditions associated with the third state comprises (i) the upper bound being greater than or equal to a threshold and (ii) the lower bound being less than or equal to the threshold.

9. The system of claim 1, wherein to obtain the measured glucose data, the processor is configured to obtain the measured glucose data from a storage system, the measured glucose data comprising historical glucose measurements.

10. The system of claim 1, wherein to obtain the measured glucose data, the processor is configured to receive the measured glucose data from a continuous analyte sensor worn by the host, the measured glucose data comprising real-time glucose measurements.

11. The system of claim 1, wherein performing the filtering operation comprises applying a centered median filter on the measured glucose data.

12. A method comprising:
obtaining measured glucose data of a host;
determining a subset of the measured glucose data, based on performing a filtering operation on the measured glucose data;
determining, for each value within the subset of the measured glucose data, a respective range of a likelihood of an occurrence of a metabolic event;
determining, for at least one value within the subset of the measured glucose data, a state of the metabolic event based in part on at least one of an upper bound or a lower bound of the respective range corresponding to the at least one value within the subset of the measured glucose data; and
causing an output indicative of the state of the metabolic event to be displayed via a display device associated with the host.

13. The method of claim 12, wherein determining the state of the metabolic event comprises:
determining whether a respective set of conditions associated with each of a first state, a second state, or a third state is satisfied; and
setting the state of the metabolic event to one of the first state, the second state, and the third state based on which respective set of conditions is satisfied.

14. The method of claim 13, wherein:
the first state is associated with presence of the metabolic event; and
the set of conditions associated with the first state comprises the upper bound of the respective range being less than a first threshold.

15. The method of claim 14, further comprising determining at least one of a distance parameter or a density parameter, based on the subset of the measured glucose data, wherein the set of conditions associated with the first state further comprises at least one of (i) the distance parameter being greater than a second threshold or (ii) the density parameter being greater than a third threshold.

16. The method of claim 15, wherein the distance parameter is a distance between (i) a first value within the subset of the measured glucose data that is below the first threshold and (ii) a second value within the subset of the measured glucose data that is below the first threshold.

17. The method of claim 15, wherein the density parameter comprises a number of values within the subset of the measured glucose data that is below the first threshold within a predetermined time period.

18. The method of claim 13, wherein:
the second state is associated with an absence of the metabolic event; and
the set of conditions associated with the second state comprises the lower bound being greater than a threshold.

19. The method of claim 13, wherein:
the third state is associated with a presence of the metabolic event and an absence of the metabolic event being undetermined; and
the set of conditions associated with the third state comprises (i) the upper bound being greater than or equal to a threshold and (ii) the lower bound being less than or equal to the threshold.

20. A non-transitory computer-readable storage medium comprising computer-executable code, which when executed by one or more processors, perform an operation comprising:
obtaining measured glucose data of a host;
determining a subset of the measured glucose data, based on performing a filtering operation on the measured glucose data;
determining, for each value within the subset of the measured glucose data, a respective range of a likelihood of an occurrence of a metabolic event;
determining, for at least one value within the subset of the measured glucose data, a state of the metabolic event based in part on at least one of an upper bound or a lower bound of the respective range corresponding to the at least one value within the subset of the measured glucose data; and
causing an output indicative of the state of the metabolic event to be displayed via a display device associated with the host.
